(19) United States
(12) Patent Application Publication
(10) Pub. No.: US 2023/0323380 A1
(43) Pub. Date: Oct. 12, 2023

(54) Phosphorus-Efficient and High Yield Gene of Crops, and Application Thereof

(71) Applicant: CAS Center for Excellence in Molecular Plant Sciences, Shanghai (CN)

(72) Inventors: Zuhua He, Shanghai (CN); Bin Ma, Shanghai (CN); Qun Li, Shanghai (CN)

(21) Appl. No.: 18/147,781

(22) Filed: Dec. 29, 2022

Related U.S. Application Data
(63) Continuation-in-part of application No. PCT/CN2021/104527, filed on Jul. 5, 2021.

(30) Foreign Application Priority Data
Jul. 7, 2020 (CN) 202010644962.6

(51) Int. Cl. C12N 15/82; C12Q 1/6895

(52) U.S. Cl. CPC C12N 15/8262; C12Q 1/6895; C12Q 2600/13

(57) ABSTRACT

Provided are a phosphorus-efficient and high-yield gene of crops, and an application thereof. It is first disclosed that a PHO1;2 gene has a regulating function on filling of crop kernels. Up-regulating the expression of the gene in crops can significantly promote filling of the crop kernels, increase the kernel weight of the crop kernels, the kernels per spike, the tiller number and the kernel thickness, and/or promote thickening of the crops. The PHO1;2 gene plays a two-way phosphorus transport effect which mainly conveys phosphorus to the extracellular domain, can regulate intracellular phosphorus accumulation, increase the utilization rate of phosphorus in crops, and improve the duration of crops to a low-phosphorus environment.

### RELATED APPLICATIONS

This application is a National Stage Application under 35 U.S.C. 371 of co-pending PCT application PCT/CN2021/104527 designating the United States and filed Jul. 5, 2021; which claims the benefit of CN application number 202010644962.6 and filed Jul. 7, 2020, each of which are hereby incorporated by reference in their entireties.

### TECHNICAL FIELD

The disclosure relates to the field of botany and molecular biology, in particular, the disclosure relates to a phosphorus-efficient and high-yield gene of crops, and application thereof.

### SEQUENCE LISTING

The instant application contains a Sequence Listing which has been submitted electronically in XML format and is hereby incorporated by reference in its entirety. Said XML copy, created on Jun. 8, 2023, is named “Sequence_Listing_008703.00032_ST26” and is 8 KB in size.

### BACKGROUND

As the population expands and the area of arable land decreases, how to grow food more efficiently on limited arable lands has always been the research focus of researchers. Traditional breeding methods can no longer meet this demand. The comprehensive use of a variety of molecular biology and molecular marker-assisted breeding methods can help people maximize crop yields. Therefore, it is very important to study the means for adjusting plant types of crops and optimizing the planting of crops.

Gramineous plants, especially rice, are main food crops in the world. Rice is also the main food for Chinese residents and important export agricultural products. As one of the most important grain crops in the world, rice has become an important research material for scientific and technological workers in recent years. Rice is China's largest grain crop, providing an important source of food for the vast majority of China's population and more than half of the world's population. However, recorded in reports, from 2005 to 2050, according to current estimates of human needs, crop yields would have to increase by 100% to meet needs of human by 2050. To study the mechanism of rice quality and genetic characteristics of rice from a molecular perspective is beneficial to provide a theoretical and practical guidance for the selection and breeding of high-quality rice.

Use of large amounts of chemical fertilizers and the deterioration of the growing environment have become big challenges to the goals of agricultural production. Therefore, on the basis of various existing studies, it is imminent to find new yield-increasing factors and maintain the development of green food. In the 1950s and 1960s, the discovery and promotion of semi-dwarf varieties brought the first green revolution to world food production. The semi-dwarf gene sd1 has been widely used in production, increasing the resistance of rice plants to lodging and fertilizer. By 2018, Li et al. reported a new green revolution triggered by the efficient utilization of N mediated by GRF4, which provided an important guarantee for sustainable development of food in the world. However, there is little research on phosphorus, another nutrient element with a large amount, especially researches on genes and molecular mechanisms to control the efficient use of phosphorus are still limited. Considering that phosphorus rock used for the production of phosphorus fertilizer is a non-renewable resource, and a considerable part of cultivated lands in the world are phosphorus deficient, resulting in the limitation of crop yield increase to a great extent. Therefore, searching for regulatory genes with high phosphorus efficiency is crucial for crop yield improvement and sustainable development for the environment.

Grain filling is an important physiological process of growth in Gramineous crops, and the quality of grain filling will directly affect the quality and yield of rice. The filling of rice kernels, a process of transporting photosynthetic products (nutrients) to the grain, is an important factor affecting the seed setting rate, quality and final yield of seeds in rice. Thus, it is of great significance to study the regulatory mechanism of the filling of rice kernels and its influencing factors for guiding high and stable yield of rice. At present, there are few studies on genes of rice directly related to grain filling, mainly including GIF1 and OsSWEET4. GIF1 is a key gene that controls the unloading of sucrose transport in rice and ultimately affects grain filling (Wang et al., 2008). The gene encodes a cytoderm sucrose invertase, which converts sucrose to glucose and fructose. In gif1, the sucrose invertase activity of cytoderm was significantly decreased, while it significantly increased after overexpression of GIF1. It indicated that GIF1-mediated sugar unloading plays an important role in the filling of rice kernels and starch synthesis. In 2015, Davide Sosso et al. reported another grain filling gene in maize, ZmSWEET4c/OsSWEET4, which encodes a hexose transporter that mainly mediates the transport of hexose from the basal endosperm transfer layer (BETL) to seeds. The mutation of this gene resulted in severe shrinkage of the maize endosperm and abnormal grain filling. At the same time, after the gene knocked out in rice, the development of endosperm turned to be severely abnormal, with grain filling cannot proceed normally (Sosso et al., 2015). The study also showed that the gene is a downstream factor of GIF1. GIF1 is responsible for the transport and disintegration of sucrose (disaccharide) into monosaccharides and OsSWEET4 is responsible for transporting monosaccharides into the endosperm for its development. Interestingly, both genes, GIF1 and SWEET4, were selected during domestication, suggesting the importance of the physiological process of grain filling.

Therefore, there is a need in this field for further researches and developments of genes related to increase crop yields, especially genes that regulate the grain filling of plants, in order to grow crops more efficiently and increase the yield of crops in per unit area.

### SUMMARY OF THE DISCLOSURE

The purpose of the present disclosure is to provide a phosphorus-efficient and high-yield gene of crops, and application thereof.

In a first aspect of the present disclosure, there is provided a method for improving crop traits or preparing crops with improved traits, comprising: up-regulating the expression or activity of PHO1;2 in crops; the PHO1;2 comprises homologues thereof; wherein, the improved crop traits are selected from the group comprising: (i) promoting filling of crop kernels (seeds); (ii) increasing crop yield or biomass, (iii) promoting two-way phosphorus transport effect which mainly conveys phosphorus to the extracellular, regulating intracellular phosphorus accumulation; (iv) enhancing ADP pyrophosphorylase (AGPase) activity; (v) increasing the utilization rate of phosphorus in crops (thus reducing the demand of crops for phosphorus fertilizer); (vi) improving the tolerance of crops to a low-phosphorus environment.

In a preferred embodiment, the up-regulation of the expression or activity of PHO1;2 comprises: overexpressing exogenous PHO1;2 in crops; preferably, it comprises: introducing a PHO1;2 gene or an expression construct or vector comprising the gene into the crops; using an enhancer or a tissue-specific promoter to improve the expression of PHO1;2 gene in crops; increasing PHO1;2 gene expression in crops with enhancers; decreasing histone-methylation level of the PHO1;2 gene and increasing its expression level; or screening varieties with high expression of gene PHO1;2 in various varieties of rice, and introducing fragments of the gene into other varieties by cross-breeding.

In another preferred example, the method also comprises up-regulating the expression or activity of GIF1 in crops.

In another preferred example, the up-regulation of the expression or activity of GIF1 comprises: introducing a GIF1 gene or an expression construct or vector comprising the gene into the crops; using an enhancer or a tissue-specific promoter to improve the expression of GIF1 gene in crops; or increasing GIF1 gene expression in crops with enhancers.

In another preferred embodiment, the tissue-specific promoters comprise (but are not limited to): nucleolar epidermis (NE) and vascular bundle (Vb)-specific promoters, and membrane-specific promoters.

In another aspect of the present disclosure, there is provided a use of PHO1;2 or an up-regulator thereof for: (a) improving the traits of crops, (b) preparing crops with improved traits, or (c) preparing formulations or compositions for improving crop traits; wherein, the improved crop traits comprise: (i) promoting filling of crop kernels (seeds); (ii) increasing crop yield or biomass, (iii) promoting two-way phosphorus transport effect which mainly conveys phosphorus to the extracellular, regulating intracellular phosphorus accumulation; (iv) enhancing ADP pyrophosphorylase activity; (v) increasing the utilization rate of phosphorus in crops (thus reducing the demand of crops for phosphorus fertilizer); (vi) improving the tolerance of crops to a low-phosphorus environment; the PHO1;2 comprises homologues thereof.

In another preferred embodiment, the formulations or compositions comprise agricultural formulations or compositions.

In another preferred embodiment, the up-regulators comprise: an expression cassette or expression construct (eg. an expression vector) overexpressing PHO1;2; or an up-regulator that interacts with PHO1;2 to increase its expression or activity.

In another aspect of the present disclosure, there is provided a crop cell, wherein, it expresses an expression cassette of exogenous PHO1;2 or homologues thereof; preferably, the expression cassette comprises: a promoter, an encoding gene of PHO1;2 or its homologues, a terminator; preferably, the expression cassette is included in a construct or an expression vector.

In another preferred embodiment, the increase of crop yield or biomass comprises: increasing grain weight, tiller number, grain number and grain thickness, and/or promoting thickening of the crops.

In another preferred embodiment, the two-way phosphorus transport effect which mainly conveys phosphorus to the extracellular comprises extracellular phosphorus transport and intracellular phosphorus transport (excluding one-way phosphorus transport).

In another preferred embodiment, the two-way phosphorus transport effect which mainly conveys phosphorus to the extracellular comprises: promoting the redistribution and recycling of phosphorus; more preferably, it comprises transferring the extra intracellular phosphorus of the crop kernels out of the endosperm cells to maintain the stability and balance of phosphorus in seeds.

In another preferred embodiment, the phosphorus is inorganic phosphorus.

In another preferred embodiment, the low-phosphorus environment refers to: compared with the normal phosphorus environment required by crops, the content of phosphorus that can be provided is reduced by 5%, 10%, 15%, 20%, 30%, 40%, 50%, 60%, 80% or 99%.

In another preferred embodiment, the “two-way phosphorus transport effect which mainly conveys phosphorus to the extracellular” means that according to the statistical analysis of transport activity, the activity of phosphorus transporting to the extracellular is significantly stronger than (for example, extracellular phosphorus transport is more than 50%, 60%, 70%, 80% of total phosphorus transports) that of intracellular transport.

In another preferred embodiment, the crops are cereal crops or the PHO1;2 or homologues thereof are derived from cereal crops; preferably, the cereal crops comprise Gramineous plants; more preferably, comprising: rice (Oryza sativa), maize (Zea mays), millet (Setaria italica), barley (Hordeum vulgare), wheat (Triticum aestivum), millet (Panicum miliaceum), broomcorn (Sorghum bicolor), rye (Secale cereale), oats (Avena sativa L), and so on.

In another preferred embodiment, the PHO1;2 comprises cDNA sequence, genomic sequence, or artificially optimized or modified sequences on the basis of them.

In another preferred embodiment, the rice is selected from the group consisting of: indica rice and japonica rice.

In another preferred embodiment, the amino acid sequence of PHO1;2 polypeptide is selected from the following groups: (i) a polypeptide having the amino acid sequence shown in any one of SEQ ID NO: 1-3; (ii) a polypeptide derived from the polypeptide of (i) by substitution, deletion or addition of one or several (such as 1-20, 1-10, 1-5, 1-3) residues in the amino acid sequence of any one of SEQ ID NO: 1-3 and having the function of regulating said traits; (iii) a polypeptide having the amino acid sequence with more than 80% (preferably more than 85%, 90%, 95% or 98%) identity to the amino acid sequence of any one of SEQ ID NO: 1-3 and having the function of regulating said traits; (iv) an active fragment of the polypeptide having the amino acid sequence shown in any one of SEQ ID NO: 1-3; or (v) a polypeptide derived from the amino acid sequence shown in any one of SEQ ID NO: 1-3 with a tag or an enzyme-cleavage sequence added at N-terminus or C-terminus; or a signal polypeptide fused at N-terminus.

In another preferred example, the amino acid sequence of GIF1 polypeptide is selected from the following groups: (i) a polypeptide having the amino acid sequence shown in SEQ ID NO: 4; (ii) a polypeptide derived from the polypeptide of (i) by substitution, deletion or addition of one or several (such as 1-20, 1-10, 1-5, 1-3) amino acid residues in the amino acid sequence of SEQ ID NO: 4 and having the function of regulating traits; (iii) a polypeptide having the amino acid sequence with more than 80% identity to the amino acid sequence of SEQ ID NO: 4 and having the function of regulating traits; (iv) an active fragment of the polypeptide having the amino acid sequence shown in SEQ ID NO: 4; or (v) a polypeptide derived from the amino acid sequence shown in SEQ ID NO: 4 with a tag or an enzyme-cleavage sequence added at N-terminus or C-terminus; or a signal polypeptide fused at N-terminus.

In another aspect of the present disclosure, there is provided a use of PHO1;2 gene or the encoded protein thereof, as a molecular marker for identifying traits of crops, or as a molecular marker for directional screening of crops; the traits comprise: (i) filling of crop kernels (seeds); (ii) yield or biomass of crops; (iii) phosphorus transport or intracellular phosphorus accumulation; (iv) ADP pyrophosphorylase activity; (v) utilization rate of phosphorus in crops; wherein, the PHO1;2 gene or the encoded protein thereof comprises homologues thereof.

In another preferred embodiment, the identification of crop traits or the directed screening can be carried out by analyzing the expression of PHO1;2 gene or the activity of PHO1;2 protein in crops.

In another aspect of the present disclosure, there is provided a method for identifying traits of crops, comprising: analyzing PHO1;2 gene expression or PHO1;2 protein activity in crops; if the PHO1;2 gene expression or PHO1;2 protein activity in crops to be tested is equal to or higher than the average value of the crops, it indicates that the crops have excellent traits, wherein the excellent traits are selected from: (i) high kernels (seeds) filling level; (ii) high yield or biomass, (iii) excellent two-way phosphorus transport effect which mainly conveys phosphorus to the extracellular, regulating intracellular phosphorus accumulation; (iv) enhanced ADP pyrophosphorylase activity; (v) increased utilization rate of phosphorus in crops; (vi) improved tolerance of crops to a low-phosphorus environment; if the PHO1;2 gene expression or PHO1;2 protein activity in crops to be tested is lower to the average value of the crops, it indicates that traits of the crops are not ideal.

In another aspect of the present disclosure, there is provided a method for directional screening crops with improved traits, the method comprises: analyzing PHO1;2 gene expression or PHO1;2 protein activity in crops; if the PHO1;2 gene expression or PHO1;2 protein activity in crops to be tested is higher than the average value of the crops, it has: (i) high kernels (seeds) filling level; (ii) high yield or biomass, (iii) excellent two-way phosphorus transport effect which mainly conveys phosphorus to the extracellular, regulating intracellular phosphorus accumulation; (iv) enhanced ADP pyrophosphorylase activity; (v) increased utilization rate of phosphorus in crops; (vi) improved tolerance of crops to a low-phosphorus environment; wherein, the PHO1;2 gene comprises homologues thereof.

In another aspect of the present invention, the method for directional screening crops with improved traits also comprises: analyzing GIF gene expression or GIF protein activity in crops; if the GIF gene expression or GIF protein activity in crops to be tested is higher than the average value of the crops, it indicates that the crops have improved traits.

In another preferred embodiment, the crop PHO1;2 gene is highly expressed or the PHO1;2 protein is at a highly activity; preferably, the high expression or high activity refers to a statistically significant increase in expression or activity compared to the average expression or activity of the same or similar crops.

In another preferred embodiment, the increase, enhancement or improvement represents significant increase, enhancement or improvement, such as increase, enhancement or improvement by 20%, 40%, 60%, 80%, 90% or higher.

In another aspect of the present disclosure, there is provided a method for screening substances (potential substances) for improving crop traits, wherein the method comprises: (1) adding candidate substance to the system expressing PHO1;2; (2) detecting the system to observe the expression or activity of PHO1;2; if the expression or activity is up-regulated, then the candidate substance can be used as the substance to improve traits of crops; wherein, the improved crop traits are selected from the following group comprising: (i) promoting filling of crop kernels (seeds); (ii) increasing crop yield or biomass, (iii) promoting two-way phosphorus transport effect which mainly conveys phosphorus to the extracellular, regulating intracellular phosphorus accumulation; (iv) enhancing ADP pyrophosphorylase activity; (v) increasing the utilization rate of phosphorus in crops (thus reducing the demand of crops for phosphorus fertilizer); (vi) improving the tolerance of crops to a low-phosphorus environment.

In another preferred embodiment, a control group is also included, so as to clearly distinguish the difference between the expression or activity of PHO1;2 in testing group and in control group.

In another preferred embodiment, the candidate substances include (but are not limited to): regulators (such as up-regulators, constructs for gene-editing, small-molecule compounds, etc) designed for PHO1;2 genes or encoded proteins thereof or upstream or downstream proteins or genes.

In another preferred embodiment, the crops are gramineous plants, or the PHO1;2 or homologues thereof are derived from gramineous plants; more preferably, comprising: rice (Oryza sativa), maize (Zea mays), millet (Setaria italica), barley (Hordeum vulgare), wheat (Triticum aestivum), millet (Panicum miliaceum), broomcorn (Sorghum bicolor), rye (Secale cereale), oats (Avena sativa L), and so on.

Other aspects of the present disclosure will be apparent to those skilled in the art based on the disclosure herein.

### BRIEF DESCRIPTION OF THE DRAWINGS

FIG. 1a-b. Gene mapping of GAF1.

FIG. 2a-j. Phenotypic characteristics of gaf1.

FIG. 3a-h. Phenotypic characteristics of gaf1.

FIG. 4a-j. Agronomic trait analysis of CRISPR/Cas9 knockout mutant alleles.

FIG. 5a-e. OsPHO1;2 is a tissue-specific membrane transporter.

FIG. 6a-g. OsPHO1;2 is a two-way phosphorus transporter which mainly conveys phosphorus to the extracellular.

FIG. 7a-g. Accumulation of Pi inhibits the activity of amylosynthetase.

FIG. 8a-d. Overexpression of AGPase can partially complement the filling defects of ko1.

FIG. 9a-c. OsPHO1;1 and OsPHO1;3 expression patterns.

FIG. 10a-i. OsPHO1;1 and OsPHO1;3 are not involved in regulating filling of crop kernels and Pi redistribution.

FIG. 11a-g. ZmPHO1;2 in maize regulates filling of rice kernels and Pi redistribution.

FIG. 12a-i. Overexpression of OsPHO1;2 can significantly promote filling and increase rice yield.

FIG. 13a-e. Overexpression of OsPHO1;2 promotes the recycling of phosphorus.

FIG. 14a-f. Overexpression of OsPHO1;2 can significantly promote filling and increase rice yield in soil with extremely low phosphorus.

FIG. 15 a-f. Overexpression of OsPHO1;2 can significantly promote filling and increase rice yield under low phosphorus conditions.

FIG. 16a-f. Overexpression of ZmPHO1;2a in maize can significantly increase yield.

FIG. 17a-f. Overexpression of ZmPHO1;2b in maize can significantly increase yield.

FIG. 18. Simultaneous overexpression of ZmPHO1;2a and ZmPHO1;2b in maize can significantly increase yield to a great extent.

### DETAILED DESCRIPTION

Based on researches of genetics and molecular biology, the inventors found that a PHO1;2 gene has the ability to regulate the filling of crop kernels. Up-regulating the expression of the gene in crops can significantly promote filling of the crop kernels, increase the grain weight of the crop kernels, the grain number per panicle, the tiller number, the grain thickness, and/or promote thickening of the crops. The inventors also found that the PHO1;2 gene plays a two-way phosphorus transport effect which mainly conveys phosphorus to the extracellular, regulating intracellular phosphorus accumulation, increasing the utilization rate of phosphorus in crops, and improving the tolerance of crops to a low-phosphorus environment The disclosure provides a new way for the improvement of cereal crops and also provides a new idea for reducing the application of natural phosphorus fertilizer and meliorating soil environment.

PHO1;2 or GIF1

As used herein, the “PHO1;2 gene or PHO1;2 protein (polypeptide)” refers to a PHO1;2 gene or PHO1;2 protein from Oryza sativa or Zea mays that is homologous to a Oryza sativa-derived or Zea mays-derived gene or polypeptide, with substantially the same structural domains and substantially the same functions.

As used herein, the “GIF gene or GIF protein (polypeptide)” refers to a GIF gene or GIF protein from Oryza sativa or Zea mays that is homologous to a Oryza sativa-derived or Zea mays-derived gene or polypeptide, with substantially the same structural domains and substantially the same functions.

In the present disclosure, the PHO1;2 protein or the GIF protein also comprise its fragments, derivatives and analogs. As used herein, the term “fragment”, “derivative” or “analog” refers to a protein fragment that essentially maintains the functions or activities of the polypeptides, and may be a protein (i) substituted by one or more conservative or non-conservative amino acid residues (preferably conservative amino acid residues), and such substituted amino acid residues may or may not be encoded by the genetic code, or (ii) with a substitution group in one or more amino acid residues, or (iii) formed by an additional amino acid sequence fused to the protein sequence, and so on. According to the teaching herein, these functional fragments, derivatives, and analogues belong to the common knowledge to those skilled in the art. Biologically active fragments of the PHO1;2 proteins or GIF proteins can all be applied to the present disclosure.

In the present disclosure, the “PHO1;2 protein” refers to a protein with the sequence shown in any one of SEQ ID NO: 1-3, which has the activity of promoting filling of crop kernels and improving crop yield. The term also comprises variants of any one of the sequence SEQ ID NO: 1-3 with the same function as the polypeptide. The “GIF protein” refers to a protein with the sequence shown in SEQ ID NO: 4 and the variants of the sequence SEQ ID NO: 4 with the same function as the polypeptide. The variants may include (but are not limited to): deletion, insertion and/or substitution of one or more (usually 1-50, preferably 1-30, more preferably 1-20, most preferably 1-10, even more preferably 1-8,1-5) amino acids, and addition or deletion of one or several (usually within 20, preferably within 10, more preferably within 5) amino acids at the C-terminal and/or N-terminal. For example, in the art, substitution with amino acids of approaching or similar properties generally does not alter the function of a protein. For another example, the addition of one or more amino acids to the C terminus and/or N terminus also generally does not alter the function of a protein.

In the present disclosure, the “PHO1;2” or “GIF1” also includes its homologues. It should be understood that although PHO1;2 or “GIF1” obtained from a specific species of Oryza sativa or Zea mays is preferred in the present disclosure, other proteins that are homologous to the PHO1;2 or “GIF1” protein (such as 80% or more, more preferably 85% or more, such as 90%, 95%, 98% or 99% homologous to the PHO1;2 or “GIF1” protein with the polypeptide sequences shown in SEQ ID NO: 1-3), with substantially the same functions, are also included in the present disclosure. Methods and tools for aligning sequence identity are also well known in the art, such as BLAST. “Homology” refers to the similarity (ie, sequence similarity or identity) between two or more nucleic acids or polypeptides in terms of certain percentage of amino acid residues in the same positions.

Proteins obtained from other species except Oryza sativa or Zea mays that are in high homologous to the polypeptide with sequences shown in SEQ ID NO: 1-4, or with substantially the same or similar functions in the same or similar regulatory pathways, are also included in the present disclosure.

The present disclosure also includes polynucleotides (genes) encoding the polypeptides, which may be natural genes from crops or their degenerate sequences.

Vectors comprising the coding sequences, as well as host cells genetically engineered from the coding sequences of the vectors or polypeptides, are also included in the present disclosure. Methods known to those skilled in the art can be used to construct suitable expression vectors.

Host cells are usually plant cells. For transforming plants, methods such as Agrobacterium transformation or biolistic transformation can generally be used, such as leaf disk method, rice immature embryo transformation method, and so on; preferably Agrobacterium transformation. Transformed plant cells, tissues or organs can be regenerated into plants using conventional methods to obtain plants with altered traits relative to the wild type.

As used herein, the term “crop” refers to a plant with economic value in agriculture and industry such as grain, cotton, oil, and so on. The economic value can be reflected in seeds, fruits, roots, stems, leaves and other useful parts of the plants. The crops include but not limited to: monocotyledonous plants or dicotyledonous plants. Preferred monocotyledonous plants are gramineous plants; more preferably rice, wheat, barley, maize, broomcorn and the like. Preferred dicotyledonous plants include but not limited to: malvaceae cotton plants, cruciferous brassica plants and the like; more preferably cotton, rape and the like.

In the present disclosure, the crops include plants expressing PHO1;2, preferably also expressing GIF1; preferably cereal crops. Preferably, the cereal crops are crops with kernels, and filling of crop kernels is involved in the development and growth of kernels. The “cereal crops” may be gramineous plants or awny plants (crops). Preferably, the gramineous plants are rice, barley, wheat, oats, rye, maize, broomcorn, and so on. The awny plants are plants that have needles on their seed shells.

The Application

Inorganic phosphorus (Pi) is an essential nutrient for plant growth and crop yield. Generally, starch synthesis in crops requires optimal levels of Pi to regulate filling of crop kernels. However, the regulatory mechanism of Pi balance in crop kernels, especially in endosperm cells, is still unclear in the prior art. In researches of the inventors, a mutant gaf1 (grain alive embryo and incomplete filling 1) with severe defects in starch synthesis and filling of crop kernels was successfully screened and obtained, and its regulatory gene GAF1 was successfully cloned by map-based cloning, encoding a phosphate transporter OsPHO1;2. Studies have shown that GAF1/OsPHO1;2 is a plasma membrane-localized phosphorus transporter with strong efflux activity, which is specifically expressed in the nucellar epidermis and ovular vasculature of seeds, and mainly regulates the redistribution of Pi and filling of crop kernels during the filling stage. After mutation, the Pi in seeds accumulated significantly, resulted in inhibiting the activity of AGPase, the key rate-limiting enzyme of starch synthesis, and leading to the inhibition of starch synthesis. Overexpression of AGPase gene could partially restore the defective filling phenotype of the mutant. In addition, in knockout transgenic maize, it was found that the homologous gene ZmPHO1;2 of OsPHO1;2 also regulates filling of kernels and Pi allocation and utilization in maize by the same functional mechanism. Field experiments showed that overexpression of OsPHO1;2 could promote filling of kernels and ultimately significantly increase yield of plants without increasing total phosphorus in seeds, especially under low phosphorus conditions, OsPHO1;2 could increase yield by the intake of low phosphorus, with high phosphorus utilization efficiency. Therefore, the inventors have successfully identified the PHO1-type phosphorus transporter, which is closely related to the filling of crop kernels and high phosphorus utilization, providing an excellent target gene for improving crop yield with minimal phosphorus fertilizer in the future.

The inventors discovered for the first time that OsPHO1;2 is a two-way phosphorus transporter (mainly conveys phosphorus to the extracellular) rather than a one-way phosphorus transporter, which is a significant discovery. For phosphorus transport, such studies were often researched in the seedling period of plants. However, in this field, it has not been found that in matured plants, such as in the filling of crop kernels, OsPHO1;2, with two-way phosphorus transport function which mainly conveys phosphorus to the extracellular, balances the phosphorus inside and outside the cells, making it possible for the reasonable redistribution of phosphorus in crops. Large amounts of Pi are required for kernel development but excessive Pi accumulation can be detrimental. Therefore, the balance of Pi supply and demand during seed development is particularly important. Although it has been shown that OsPT4, OsPT8 and SPDT are involved in the distribution and transport of Pi in seeds, there is no research on how Pi is unloaded in seeds. On the basis of kernel filling, balancing phosphorus transport from source to sink and redistribution/retransport from sink to source is the key to phosphorus redistribution among different tissues, which determines phosphorus utilization efficiency (PUE) in plants. Therefore, studying the mechanism of phosphorus redistribution and recycling process will help to understand the link between grain filling/yield and PUE, which is necessary for guiding the increase of crop yield and the efficiency of phosphorus fertilizer utilization, and the reduction of phosphorus fertilizer to achieve green and sustainable agricultural development.

Based on the new findings of the inventors, there is provided a method for improving plant traits or preparing plants with improved traits, comprising: up-regulating the expression or activity of PHO1;2 in plants; the PHO1;2 comprises homologues thereof; wherein, the improved traits are selected from the group comprising: (i) promoting filling of crop kernels (seeds); (ii) increasing crop yield or biomass, (iii) promoting two-way phosphorus transport effect which mainly conveys phosphorus to the extracellular, regulating intracellular phosphorus accumulation; (iv) enhancing ADPase activity; (v) increasing the utilization rate of phosphorus in crops; (vi) improving the tolerance of crops to a low-phosphorus environment. Preferably, it further comprises up-regulating the expression or activity of GIF1 in plants.

It should be understood that according to the experimental data and regulatory mechanisms provided by the present disclosure, various methods well known to those skilled in the art can be used to regulate the expression of the PHO1;2 or GIF1, and these methods are all included in the present disclosure.

In the present disclosure, substances that up-regulate the expression or activity of PHO1;2 or GIF1 in plants include promoters, agonists, activators and up-regulators. The “up-regulation”, “improvement” or “promotion” includes “up-regulation”, “promotion” of protein activities or “up-regulation”, “improvement” and “promotion” of protein expressions. Any substance that can increase the activity of PHO1;2 or GIF1 protein, increase the stability of PHO1;2 or GIF1 gene or the encoded protein thereof, upregulate the expression of PHO1;2 or GIF1 gene and increase the effective time of PHO1;2 or GIF1 protein can be used in the present disclosure as useful substances for up-regulating PHO1;2 or GIF1 genes or the encoded proteins thereof. They can be chemical compounds, small chemical molecules, biomolecules. The biomolecules can be nucleic acids (including DNA, RNA) or proteins.

As another example of the present disclosure, there is also provided a method for up-regulating the expression of PHO1;2 or GIF1 genes or encoded proteins thereof in plants, wherein the method comprising: transferring PHO1;2 or GIF1 genes, constructs or vectors of the encoding protein thereof into the plants, obtaining plant tissues, organs or seeds transformed by PHO1;2 or GIF1 encoding polynucleotides; and obtaining the plants after the regeneration of the plant tissues, organs or seeds encoding polynucleotides with exogenous PHO1;2 or GIF1.

Other methods for increasing gene expression of the PHO1;2 or GIF1 or homologues thereof are known in the art. For example, the gene expression of the PHO1;2 or GIF1 or homologues thereof can be enhanced by a strong promoter. Alternatively, the gene expression of the PHO1;2 or GIF1 can be enhanced by an enhancer (eg. the first intron of the rice Waxy, the first intron of the Actin, etc.). Strong promoters suitable for the method of the present disclosure include but are not limited to: 35S, Ubi of rice and maize, and so on.

The methods can be carried out using any suitable conventional means, including reagents, temperature, pressure conditions, and the like.

Based on the function of PHO1;2 or GIF1 genes, the genes can be used as molecular markers to directionally-screen plants. Based on this new discovery, it is also possible to directionally-screen for substances or potential substances that regulate plant types, yields, organelles or cell cycles by modulating this mechanism. PHO1;2 or GIF1 or its encoded protein can also be used as a tracking marker for genetically transformed plants.

Therefore, in the present disclosure, there is provided a method for directional screening or identifying plants, the method comprises: analyzing PHO1;2 or GIF1 gene expression or activity in plants to be tested; if the PHO1;2 or GIF1 gene is highly expressed or is highly active in the testing plants, then the plants have: (i) high kernels (seeds) filling level; (ii) high yield or biomass, (iii) excellent two-way phosphorus transport effect which mainly conveys phosphorus to the extracellular, regulating intracellular phosphorus accumulation; (iv) enhanced ADPase activity; (v) increased utilization rate of phosphorus; (vi) improved tolerance of crops to a low-phosphorus environment; with improved traits; otherwise, the traits of the plants are not ideal. Preferably, it also comprises identifying GIF gene expression or activity in plants to be tested.

When analyzing the plants to be tested, the expression or mRNA level of PHO1;2 can be determined to know whether the expression or mRNA level in plants to be tested is higher than the average value of such plants. If it is significantly higher, it has improved traits.

In the present disclosure, there is provided a method for screening types, yields, organelles or cell cycles of plants, the method comprises: adding candidate substance to the system expressing PHO1;2; detecting the system to observe the expression or activity of PHO1;2; if the expression or activity is up-regulated, then the candidate substance can be used to influence plant traits and lead to (i) high kernels (seeds) filling level; (ii) high yield or biomass, (iii) excellent two-way phosphorus transport effect which mainly conveys phosphorus to the extracellular, regulating intracellular phosphorus accumulation; (iv) enhanced ADPase activity; (v) increased utilization rate of phosphorus; (vi) improved tolerance of crops to a low-phosphorus environment. Preferably, the system also expresses GIF1 and by detecting the system, whether the expression of GIF1 is up-regulated is to be determined.

The methods for screening substances acting on a protein or gene or its specific region as a target are well known to those skilled in the art, and these methods can be used in the present disclosure. The candidate substances can be selected from: peptides, polymeric peptides, peptidomimetics, non-peptide compounds, carbohydrates, lipids, antibodies or antibody fragments, ligands, small organic molecules, small inorganic molecules, nucleic acid sequences, and the like. Based on the type of substances to be screened, it is clear to those skilled in the art how to select a suitable screening method.

The detection of protein-protein interactions and the strength of the interactions can be performed using a variety of techniques well-known to those skilled in the art, such as GST sedimentation (GST-Pull Down), bimolecular fluorescence complementation assay, yeast two-hybrid system or Immunoprecipitation, and so on.

After large-scale screening, a class of potential substances that specifically act on PHO1;2 and have regulatory effects on plant types, yields, organelles or cell cycles can be obtained.

The disclosure if further illustrated by the specific examples described below. It should be understood that these examples are merely illustrative, and do not limit the scope of the present disclosure. The experimental methods without specifying the specific conditions in the following examples generally used the conventional conditions, such as those described in J. Sambrook, Molecular Cloning: A Laboratory Manual (3^{rd} ed. Science Press, 2002) or followed the manufacturer's recommendation.

Experimental Materials

1. Genetic Material and Phenotype Analysis

The rice mutant grain aberrant and incomplete filling 1 (gaf1) is a natural mutant selected from the field germplasm database (from Zhejiang Academy of Agricultural Sciences). Gaf1 was crossed with wild-type Zhenshan 97 (ZS97) to obtain F1, and F1 was self-crossed to obtain F2, resulting in an F2 mapping population, which was used for the initial mapping of gaf1. In the F1 population crossed with Nipponbare (NIP), a single plant was selected and backcrossed with Nipponbare to obtain BC1F1, and then the molecular marker linked to the phenotype in the initial mapping was used to identify the individual plant containing the recessive locus of gaf1. Nipponbare was used as backcross parents to obtain BC2F1, and then through the identification and screening of molecular markers on both sides of the initial positioning, after carefully observing the grain filling phenotype in BC3F2, the line gaf1 with incomplete filling and the wild type line GAF1 lead to a pair of close isogenic lines, namely NIL-GAF1 (for Nipponbare, NIP background, GAF1 wild type), NIL-gaf1 (for Nipponbare, NIP background, GAF1 mutant), were used for fine mapping and phenotyping.

All rice transgenic materials were based on wild-type NIP or the mutant ko1 (obtained from Nipponbare, NIP background, GAF1/OsPHO1;2 gene knockout material), and transgenic lines were generated by Agrobacterium EHA105-mediated genetic transformation, with T1-T3 generation of homozygous lines used for phenotypic analysis. All rice materials were grown in Shanghai Songjiang (summer) and Hainan Lingshui (winter).

Transgenic maize, with the inbred line C01 and B104 (obtained from China Seed Company and Changzhou Weimi Company, commonly used inbred lines for maize genetic transformation) as the background material, was used to generate transgenic lines through the genetic transformation mediated by Agrobacterium EHA105. After obtaining the TO generation seeds, the seeds were planted in Shanghai Transgenic greenhouses in Songjiang two seasons a year and each generation were applied by strict bagged selfed-seed method for 3 consecutive generations, then homozygous lines were selected for phenotypic analysis.

After each line of rice and maize was homozygously stabled, the 1000-grain weight, 100-grain weight, seed-setting rate, grain number per panicle, the tiller number, grain length, grain width, grain thickness, plant height, yield per plant and other phenotypes or agronomic traits at maturity were observed and statistically analyzed. The tiller number was counted after the plants were fully mature, and the height was directly measured with a scaled bamboo ruler in the field, which was the distance from the ground to the highest position of the spike. The 100-grain weight, 1,000-grain weight and yield per plant are measured by electronic balance. The seed setting rate is the ratio of the number of full grains in each spike to the total number of grains. The grain thickness was measured at the middle part of the seed (the thickest part) directly with a vernier caliper. The grain length and grain width were measured by Wanshen SC-G seed tester.

2. Gene Mapping Molecular Marker Designing

The markers required for initial gene mapping are the polymorphic parts of the 500 pairs of SSR markers reserved in this laboratory. InDel primers are designed for the areas that cannot be covered. For Indel information, refer to 9311 and Nipponbare polymorphism database. The fine mapping is all dCaps markers. The dCaps 2.0 (http://helix.wustl.edu/dcaps/dcaps.ht) website is used for designing markers. Two SNPs and flanking sequences were input respectively, and a modified primer was obtained after running, with a suitable endonuclease be selected, and then Primer 5.0 was used to find another primer. The size of the amplification product was controlled between 150-300 bp.

3. Gene Expression Analysis

Plant materials such as seeds, leaves and other tissues were collected in a 2 mL imported EP tube (with steel balls added in advance), and the tube were snap-frozen in liquid nitrogen. After grinding into powder at 40 Hz and 50 s with a grinder, a TRIzol (Invitrogen) method was used to extract total RNA. 2 μg of total RNA was taken for reverse transcription according to the instructions of Weizan reverse transcription kit, and the cDNA product was used for qPCR analysis. The detection instrument were Bio-Rad real-time PCR fluorescent-quantitative instrument and SYBR® Premix Ex Taq™ (2×) (Takara). A two-step amplification procedure was used for the reactions: pre-denaturation at 95° C. for 30s, denaturation at 95° C. for 10s, annealing and extension at 60° C. for 30s, 40 cycles, with melting curve analysis added. The relative expression of genes was analyzed by 2^{-ΔΔ}^{^{CT}} method.

4. Detection of Protein Expression

A. Extraction of Total Proteins from Plant Tissues

(1) Formulation of the extracts (suitable for all tissues of rice): 50 mM Tris-HCl, pH 8.0, 0.25M sucrose, 2 mM EDTA, pH 8.0, 2 mM DTT (add before use), 1 mM PMSF (add before use); (2) 0.5 g of fresh rice tissue was taken, with 1 mL of extract added, mixed by shaking at 4° C. for 30 minutes; (3) Centrifuge at 12,000 rpm and 4° C. for 15 minutes; (4) The supernatant was removed into a new 1.5 mL EP tube; (5) Re-centrifuge for ensuring to remove impurities. The supernatant is the protein; (6) Part of the supernatant was taken, with an equal volume of 2×SDS loading buffer (+DTT) added, denatured in a boiling water bath for 5 min, and quickly cooled on ice.

B. Western Blot

(1) Prepared SDS-PAGE precast gel was taken out and rinsed with distilled water. The electrophoresis solution was added to the electrophoresis tank, with the comb be pulled out; (2) About 20-40 μL of protein sample was loaded into each well, with electrophoresis at 100V constant pressure for about 2 h; (3) Prepare for transmembrane. The membrane was cut into a suitable size, marked with a pencil, firstly soaked and activated in methanol for 15s, then shaked in H₂O for 10 minutes, and then put into wet transfer solution together with glue and soaked for 10 minutes; (4) 180 mA constant current for 2 h of transmembrane; (5) The transferred membrane was immediately blocked in 5% milk for 2 h; (6) Rinse in 1×TBST for 2×5 min; (7) Incubation with primary antibody at room temperature for 1-2 h or at 4° C. overnight; (8) Rinse in 1×TBST for 3×15 min; (9) Incubatate with secondary antibody for 1 h at room temperature; (10) Rinse in 1×TBST for 3×15 min; (11) After 200 μL ECL luminescent solution added, the results were visualized in an image scope and analyzed.

5. Subcellular Localization Observation-Protoplast Transformation

(1) The roots and leaves of rice seedlings were cut off, with leaf sheath remained. The leaf sheath was cut into 0.5-1 mm pieces with a single-sided blade, and infiltrated in 10 mL of 0.6M Mannitol to maintain the osmotic pressure; (2) After all leaf sheath be cut off, they were infiltrated for 10 minutes; (3) Mannitol was removed and 10 mL of enzymatic hydrolysis solution was added, then enzymolysis was carried out at room temperature for 4-5 hours in the dark; (4) The protoplasts were filtered with a 40 m pore filter into a new 50 mL centrifuge tube, with an equal volume of W5 (154 mM NaCl, 125 mM CaCl₂, 5 mM D-Glucose, 5 mM KCl, 2 mM MES-KOH) added to terminate the enzymolysis by shaking vigorously for 10s; (5) Centrifuge at 100 g at room temperature for 2 min (break is 0); (6) The supernatant was removed (by using a pipette tip with a cut tip), and 15 mL W5 solution was added and gently resuspended and centrifuged at 100 g for 2 min; this process was repeated once; (7) The supernatant was removed, and according to the number of transgenes, an appropriate amount of MMG (4 mM MES-KOH (pH 5.7), 0.5M mannitol, 15 mM MgCl₂) was added solution (about 1.5 mL), resuspended gently, and examined by a microscope; (8) 10 μL of plasmid DNA (1 μg/μL) was added to a 2 mL EP round-bottom centrifuge tube, then 100 μL of protoplasts was added and mixed gently, and finally 110 μL of PEG-Ca²⁺ bioconversion liquid (40% PEG 4000, 0.2M mannitol, 0.1M CaCl₂) was added, with the tube flicked by fingers, mixed, and transformed in the dark for 15 minutes; (9) After 440 μL of W5 solution was added, the tube was inverted and mixed gently to stop the reaction, with centrifugation at 100 g for 2 minutes; (10) The supernatant was removed and 1 mL of W5 solution was added to resuspend, with centrifugation at 100 g for 2 minutes; (11) 500 μL of W5 solution was added to resuspend the solution. Incubate horizontally overnight at 25° C. Take it out gently the next day for observing fluorescence with Confocal.

6. Gene Expression Analysis of Tissues

A. GUS Staining

The 3 Kb promoter upstream of the GAF1/OsPHO1;2 gene coding region was fused to the upstream region of the reporter gene GUS, and then ligated into the pCambia-1300 vector. The constructed pOsPHO1;2::GUS recombinant plasmid was transformed into rice NIP with agrobacterium, and 10 independent transgenic lines were obtained.

The tissues were put into an appropriate amount of GUS staining solution (containing 100 mM pH 7.0 sodium phosphate buffer, 10 mM EDTA, 0.1% Triton 100, 1 mM X-Gluc). After vacuum pumping, the GUS vitality of each tissue was observed and taken pictures after 24 hours of coloration at 37° C.

B. Immunofluorescence

(1) A fresh rice sample (the young root is about 14 days at the seedling stage, Node I is the heading stage, and other tissues are all acceptable) was taken, then after 4% w/v paraformaldehyde (containing 60 mM Suc and 50 mM cacodylic acid, pH 7.4) added, the tissue was fixed at room temperature for 2 h, with irregular exhaust in the middle by attention; (2) After fixation, the sample was washed 3 times with 60 mM Suc and 50 mM cacodynic acid (pH 7.4); (3) The fixed sample was embedded with 5% agar (low melting point) and sliced by a vibrating microtome, with the thickness of the sections are 80 m; (4) The sliced sections were placed on a glass slide, and the PBS buffer (10 mM PBS, pH 7.4, 138 mM NaCl, 2.7 mM KCl) containing 0.1% (w/v) pectolyase Y-23 (pectinase) was used and incubated at 30° C. for 2 h; (5) The sliced sections was changed to PBS buffer (10 mM PBS, pH 7.4, 138 mM NaCl, 2.7 mM KCl) containing 0.3% (v/v) Triton X-100 and incubated at 30° C. for 2 h; (6) The sections were washed 3 times with PBS buffer (10 mM PBS, pH 7.4, 138 mM NaCl, 2.7 mM KCl); (7) The glass slide was blocked with PBS buffer containing 5% (w/v) BSA; (8) The glass slide was incubated by primary antibody in a temperature-controlled box at 37° C. overnight. Antibody dilution ratio refers to the specific conditions, usually 1:50, 1:100, 1:500, with PBS for dilution; (9) The sections were washed 3 times with PBS buffer (10 mM PBS, pH 7.4, 138 mM NaCl, 2.7 mM KCl), then the slides were blocked with PBS buffer containing 5% (w/v) BSA; (10) The glass slide was incubated by secondary antibody at room temperature for 2 h, wherein the secondary antibody is Alexa Fluor 554 goat anti-rabbit IgG (red fluorescence); (11) The glass slide was washed 5 times with PBS buffer (10 mM PBS, pH 7.4, 138 mM NaCl, 2.7 mM KCl); (12) After adding a few drops of PBS containing 50% (v/v)glycerol, the glass slides were sealed with a cover glass; (13) Laser-scanning confocal microscope was used to observe and take pictures.

7. Sample Observation by a Scanning Electron Microscope

Since the objects are mature seeds of rice and maize, there is no need for drying and dehydration. The seeds were directly cutted horizontally in the middle by a scalpel. It is best to let the seeds collapse naturally without damaging the cross sections. Dryed in an oven at 37° C. for about a day The treated material was fixed on a copper table, coated with conductive colloid and then plated with gold (JEOL, JFC-1600), and observed by an electron microscope (JEOL, model JSM-6360LV) with the acceleration voltage 6 kV. A field emission scanning electron microscope (Zeiss) was used for part of the samples, and the copper stage and gold plating were slightly different from the above. The accelerating voltage was 5 kV.

8. Measurement of Soluble Sugar and Total Starch in Rice Tissues

Rice seeds (0.40 g) were taken, fully grinded with liquid nitrogen, put into a 2 mL centrifuge tube, with 1 mL MillQ water added. After opening the cap of the centrifuge tube, the tube was put in a 100° C. water bath for 15-20 min, and transferred to a 10 mL centrifuge tube. According to the weight of the samples, the volume was adjusted to 5-10 mL with MillQ water. Then the tube was centrifuged at 10,000 g for 10 min, with the supernatant filtered with a 0.45 m filter; The filtered clarified sample solution was manually loaded or put into a sampling bottle for automatic sampling (0.6 mL sample). Glucose, fructose and sucrose were analyzed on an ion chromatograph (ICS-3000, DIONEX) with CarboPac™ PA1 column. The mobile phase was 200 mM NaOH solution, the flow rate was 1.5 mL/min, and the electrochemical detector was used.

The rice seeds were grinded, passed through a 0.5 mm sieve and the grinded samples (accurately weigh 100 mg) were added into a tube (16×120 mm), making sure that all samples are at the bottom of the tube. 0.2 mL of ethanol solution (80% v/v) was added to wet the samples for dispersion and mixed with a vortex mixer. The total starch of the samples was measured using a Megazyme K-TSTA kit.

9. Measurement of AGP Pyrophosphorylase Activity

A. Crude Enzyme Extraction

(1) The seeds during filling stage were used and put into a steel pipe containing large steel balls immediately after shelling, freezed in liquid nitrogen, and grinded into powder with a 40 Hz 60s grinder; (2) With 50 mg powder in each tube, precooling extraction buffer (100 mM Tricine-NaOH, pH 8.0, 8 mM MgCl₂, 2 mM EDTA, 50 mM β-mercaptoethanol, 12.5% v/v glycerol, 5% w/v PvPP40) was added and mixed by vortex oscillation; (3) After that, the tube was mixed with a vortex mixer in a refrigerator at 4° C. for about 1 hour; (4) Then, the centrifuge tube was centrifuged at 10,000 g at 4° C. for 15 minutes, with the supernatant collected as the crude enzyme extract, which can be frozen in a −20° C. refrigerator for several months.

B. AGPase Enzyme Reaction

(1) The above crude enzyme extract was dispensed by 50 μL per tube, and prepared for enzyme reaction; (2) The enzyme reaction system for configuration: 100 mM HEPES-NaOH, pH 7.4, 1.2 mM ADP-glucose, 3 mM pyrophosphate, 5 mM MgCl₂, 4 mM DTT; (3) 200 μL of enzyme reaction solution was added to each tube with 50 μL of crude enzyme extract, and reacted in a 30° C. water bath for 20 minutes; (4) Immediately after the enzyme reaction was completed, the reaction was stopped by a boiling water bath for 2 minutes and a quick refrigeration on ice. (5) Centrifugate at 12000 rpm and 4° C. for 10 min, with 200 μL of supernatant added to a new 1.5 mL EP tube or microplate; (6) At this time, a microplate reader or spectrophotometer was used to record the first OD340(ΔA1); (7) After adding 30 μL of 2 mM NADP, 2 μL of 0.08U phosphoglucomutase, and 2 μL of 0.07U G6P dehydrogenase on ice and mixing immediately, the reaction was carried out again at 30° C. for 5-10 min; (8) A microplate reader or spectrophotometer was used to record the second OD340 (ΔA2); (9) The increase value of OD340 was calculated (ΔA=ΔA2−ΔA1), and the enzymatic activity of AGPase was calculated according to the formula.

10. Measurement of Phosphorus

A. Sample Preparation

The sun-dried rice seeds or other tissues were dried in an oven at 60° C. for 72 hours, then hulled with a husk remover, and the unpolished rice was pulverised into powder with a cyclone mill (UDY, USA), then passed through a 0.5 mm sieve for the measurement of total phosphorus, inorganic phosphorus and other elements.

B. The inorganic Phosphorus (Pi) Test

0.5 g of the sample was used, with 10 mL of extract (12.5% TCA+25 mM MgCl2) added. After shaking at 4° C. overnight, centrifuging at 10000 g for 15 min at 4° C., 5 mL of the supernatant was taken and the content of P was measured by ammonium molybdate spectrophotometric method. Each sample was repeated 3 times. Most of the samples can be transferred to the microtiter plate for the measurement.

Preparation of P standard solution for drawing the working curve: Potassium dihydrogen phosphate was dryed at 105° C. for 1 hour and cooled in a desiccator. 0.2195 g of the sample was weighed and dissolved in water, then transferred to a 1000 mL volumetric flask, with 3 mL of nitric acid and deionized double distilled water added to the constant volume. After shaking the mixture, 50 μg/mL P standard solution was prepared. 0.0, 1.0, 2.0, 4.0, 8.0, 16.0 mL of P standard solution was accurately weighed and transferred into a 50 mL volumetric flask, with 10 mL of ammonium vanadium molybdate chromogenic reagents (containing 100 g/l ammonium molybdate, 2.35 g/L ammonium vanadate and 165 mL/L 65% nitric acid), and deionized double distilled water diluted to the constant volume. After shaking the mixture, the mixture was placed at room temperature for 10 min. With 0.0 mL P standard solution as a control, the absorbance of various P standard solutions was measured with a 751-type spectrophotometer at a wave length of 400 nm. With the Pi content as the abscissa and the absorbance as the ordinate, the working curve (GB/T 6437-2002) was drawed.

C. The Total Phosphorus (P) Test

About 10 mg of each sample was added to a microwave digestion tube, and then 1 mL of 65% concentrated HNO₃ was added to each tube. The Microwave3000 (Anton PAAR, Graz, Austria) microwave digestion system was used to digest the sample for about 4-5 hours; The lid of the microwave digestion tube was opened and placed in an acid scavenger at 160° C. for the ascorbic acid reaction (about 1-1.5 hours); The remaining 1.0 mL is appropriate, with deionized water added to the constant volume of 14 mL. The digested samples were tested for the concentrations of P, S and other various trace elements. Total phosphorus content was measured using an inductively coupled plasma optical emission spectrometer (ICP-OES) (Optima 8000DV, PerkinElmer, USA). Six biological replicates were set up for each sample.

11. Elemental Determination of μXRF Fluorescence Micro-Area Spectrometer Mature seeds of rice or maize were dried at 37° C. for about 2 days, shelled, cut in the middle or broken by hand. The other ends of the seeds were cut flat with a single-sided blade to ensure a flat state.

Prepared sample was glued on the instrument stage with double-sided tape, and the position was adjusted so that it is in the center. The instrument used in this experiment was an X-ray fluorescence spectrometer (M4 Tornado, Bruker) from Shanghai Boyue Instrument Company. The parameters are as follows:

| | |
|---|---|
| Excitation Condition | 50 kV, 600 μA, Vacuum Path |
| Detector | Silicon Drift Detector (SDD) |
| Detector Energy Resolution | <150 eV |
| X-Ray Beam Spot Size | ≤20 μm for Mo—K |

After setting the parameters, the instrument starts to run. In this experiment, each sample needs to be scanned for about 2.5 hours, and each sample is set to repeat with 3 seeds. After the run, the original file was saved, the elemental content and imaging map were analyzed.

12. Pi Test of Plants In Vivo by ³¹P NMR

The hydroponic seedlings around two weeks and the endosperm in the early stage of grain filling were used to measure the Pi content of the internal plants. The samples must be guaranteed to be living plants and cannot be stressed. An appropriate weight of sample (about 0.05 g young root) was put into an NMR tube with a diameter of 5 mm, with the perfusate added. The lid of the tube was covered, and the tube was put into the NMR sampler for testing. The parameters are as follows:

| | |
|---|---|
| Instrument model | NMR |
| MestReNova software version | 6.1.1-6384 |
| Data Collection | 242.9-MHz lock with deuteroxide |
| pulse angle | 30° |
| scans | 1500 |
| spectral window | 16 kHz |
| run time | 2.1 h/sample |

10 mM methylenediphosphonic acid was used as ref, which is equivalent to 18.9 ppm of Pi, and the chemical shifts of the sample to be tested were calculated by ref.

13. Patch-Clamp Analysis of PHO1s Transport Activity

A. Cell Expression

Full-length CDS sequences of OsPHO1;1, OsPHO1;2, OsPHO1;3, ospho1;2 were cloned into the mammalian cell expression vector pEGFP-C1, and transformed into E. coli to screen for positive clones. Firstly, the mammalian cell line HEK293T was incubated at 37° C. in DMEM medium (Dulbecco's Modified Eagle's Medium) containing 10% BSA (5% CO₂ in the incubator) to prepare for transformed plasmid. The plasmids were then extracted by the QIAGEN Plasmid Mini Kit to improve the purity. 2 μL of each plasmid was added to a 6-well cell culture plate, and then, the cell transfection was completed by Lipofectamine™ 3000 Transfection Reagent Kit. Due to the GFP tag in the vector, positive cells were firstly screened by observing the GFP signal to continue downstream experiments.

B. Transport Activity Detection

In this experiment, the whole-cell patch clamp system was used to detect the activity and the Axopatch-200B patch clamp program was used.

Electrolyte formulation: 150 mM NMDG (N-Methyl-D-glucamine), 50 mM PO₄₃, 10 mM HEPES, pH 7.5 (adjusted with NMDG);

Electrode solution formula: 150 mM NMDG, 50 mM PO₄₃, 10 mM EGTA, 10 mM HEPES, pH 7.5 (adjusted with NMDG);

Voltage recording process: The electrode was continuously stimulated with a 100 ms step pulse, with the step voltage ranged from −180 mV to +100 mV (+20 mV in each step). After 1 minute, voltage states of all cells were recorded in HEK293T, using pClamp10.7 software to analyze the data.

14. Establishment of OsPHO1;2 Homozygous Overexpressed Line

The full-length gDNA sequence of OsPHO1;2 was amplified and ligated into the pCambia-1300::35SN overexpression vector by restriction endonuclease ligation. The wild-type NIP (obtained from Nipponbare, NIP background) was used as the background. Transgenic lines were generated by Agrobacterium EHA105-mediated genetic transformation, and the T1-T3 generation homozygous lines were used for phenotypic analysis. All rice materials were grown in Shanghai Songjiang (summer) and Hainan Lingshui (winter).

15. Gene/Protein Sequences

The amino acid sequence of OsPHO1; 2 in Oryza
sativa is as follows
(SEQ ID NO: 1):
MVKFSREYEASIIPEWKAAFVDYKRLKKLIKRIKVTRRDDSFAAANAAAA
ADHLLPPPPAEKEAGGYGFSILDPVRAIAARFSAGQQPSASEDEECPDRG
ELVRSTDKHEREFMERADEELEKVNAFYTGQEAELLARGDALLEQLRILA
DVKRILADHAAARRARGLARSRSMPPPPPSSSPPSSVHGSSGRYLLSGLS
SPQSMSDGSLELQQAQVSEGAAVADEVMAALERNGVSFVGLAGKKDGKTK
DGSGKGRGGGGGGGGGVLQLPATVRIDIPATSPGRAALKVWEELVNVLRK
DGADPAAAFVHRKKIQHAEKNIRDAFMALYRGLELLKKFSSLNVKAFTKI
LKKFVKVSEQQRATDLFSEKVKRSPFSSSDKVLQLADEVECIFMKHFTGN
DRKVAMKYLKPQQPRNTHMITFLVGLFTGTFVSLFIIYAILAHVSGIFTS
TGNSAYMEIVYHVFSMFALISLHIFLYGCNLFMWKNTRINHNFIFDFSSN
TALTHRDAFLMSASIMCTVVAALVINLFLKNAGVAYANALPGALLLLSTG
VLFCPFDIFYRSTRYCFMRVMRNIIFSPFYKVLMADFFMADQLTSQIPLL
RHMEFTACYFMAGSFRTHPYETCTSGQQYKHLAYVISFLPYFWRALQCLR
RYLEEGHDINQLANAGKYVSAMVAAAVRFKYAATPTPFWVWMVIISSSGA
TIYQLYWDFVKDWGFLNPKSKNRWLRNELILKNKSIYYVSMMLNLALRLA
WTESVMKIHIGKVESRLLDFSLASLEIIRRGHWNFYRLENEHLNNVGKFR
AVKTVPLPFRELETD
The amino acid sequence of ZmPHO1; 2a in Zea mays
is as follows
(SEQ ID NO: 2):
MAALERNGVSFVGSGLGSKAKKDGGGKQLTGRAAALPATVRIDVPPTSPG
RAALKVWEELVNVLRKDGADPAAAFVHRKKVQHAEKSIRDAFLALYRGLD
LLNKFSSLNVKAFTKILKKFVKVSEQQRKTDLFSEKVKRSPFSSSDKVLQ
LADEVECIFSRHFAGNDRKVAMKYLKPQQPRNTHMITFLVGLFTGTFVSL
FIIYSVLAHVAGIFSSTGNTAYMEIVYHVFSMFALISLHVFLYGCNLLAW
KSSRISHNFIFDFSPSTALTHRDAFLLSASIMCTVVAALVVNLFLSNAGA
TYANALPGALLLLSAAALFCPFNVFYRSTRYCFMRVMRNIMLSPFYKVLM
ADFFMADQLTSQIALLRHLEFTGCYFMAGTFTTHAYGSCTSSSQYKNLAY
VLSFLPYYWRAMQCLRRYLEEGHDIDQLANAGKYISAMVAAAVRFKYAAA
PTPFWMWMVIVSSTGATIYQLYWDFVMDWGFLDLRSKNRWLRDQLILKNK
PIYYASMMLNLVLRLAWAESVMKLRLGMVESRLLDFSLASLEIIRRGHWN
FYR
The amino acid sequence of ZmPHO1; 2b in Zea mays
is as follows
(SEQ ID NO: 3):
MVKFSREYEASIIPEWKAAFVDYKGLKKLVKRIKIARRDRAARSTSNDHD
DATTTTYGFSVLDPVRALASHFNNATPPASPEGGSDDALRSLESDSGELV
RATDKHEQEFVERADEELEKVNKFYAAQEADMLARGDALIEQLRILADVK
RILADHAAASSRRGRARLARTGGNSSPPSVDGSNSGRHLLSSPFVASSPQ
SMSDGSVQLQQARVAEGAAVAEEVMAALERNGVSFVGGGLGKAKKDGSGK
QLMGRAALLQLPATVRIDIPPTSPGRAALKVWEELVNVLRKDGADPAAAF
VHRKKVQHAEKSIRDAFLALYRGLDLLKKFSSLNVKAFTKILKKFVKVSE
QHRKGDLFSEKVKRSPFSSSDKVLQLADEVECIFLRHFAGNDRKVAMKYL
KPQQPRNTHMVTFLVGLFTGTFVSLFIIYSVLAHVAGIFSSTGNTAYMEI
VYHVLSMFALISLHVFLYGCNLSMWKGTRINHNFIFDFSSTALTHRDAFL
MSASIMCTVVAALVVNLFLRNAGATYANALPGALLLLSAGVLFCPFNIFY
RSTRFCFMRVMRNIVLSPFYKVLMADFFMADQLTSQIPLLRHLEFTGCYF
MAETFRTHAYGSCTSSSQYKNLAYVLSFLPYYWRAMQCLRRYLEEGHDMN
QLANAGKYVSAMVAAAVRFKYAATPTPFWMWMVIASSTGATIYQLYWDFV
MDWGFLNPKSKNFWLRDQLILKNKSIYYASMMLNLVLRLAWAESVMKLRL
GMVESRLLDFSLASLEIIRRGHWNFYRLENEHLNNAGKFRAVKTVPLPFR
ELETD
The amino acid sequence of ZmGIF1 in Zea mays is
as follows
(SEQ ID NO: 4):
MRALVVVSFASACLLLLLQLAGASHVVYNYKDLEAEAAAATDQVPPSIVN
PLLRTGYHFQPPKNWINDPNAPMYYKGWYHFFYQYNPKGAVWGNIVWAHS
VSRDLINWVALEPALRPSIPGDRYGCWSGSATVLPDGGGPVIMYTGVDHP
DINYQVQNVAYPKNVSDPLLREWVKPSHNPVIVPEGGINATQFRDPTTAW
RGPGPEQWRLLVGSAAGSSPRGVAYVYRSRDFRRWRRVRRPLHSAATGMW
ECPDFYPVSKGGAPRAGLETSVPPGPRVKHVLKNSLDLRRYDYYTVGTYH
PRAERYVPDDPAGDEHRLRYDYGNFYASKTFYDPAKRRRILWGWANESDS
AADDVAKGWAGIQAIPRTVWLDPSGKQLLQWPIEEVEALREKSVTLKNRL
IKAGHHVEVTGIQTAQADVEVSFEVSPAALAGAETLDPALAYDAEKLCGV
KRADVRGGVGPFGLWVLASANRKERTAVFFRVFKPAAGSDKPVVLMCTDP
TKSSLNPNLYRPTFAGFVDTDISNGKISLRSLIDRSVVESFGAGGKTCIL
SRVYPSLAIGKDARLYVENNGRAHVKVSRLTAWEMKKPVMNGA.

### Example 1. Gene Mapping and Phenotypic Analysis of Incomplete Filling Mutant gaf1

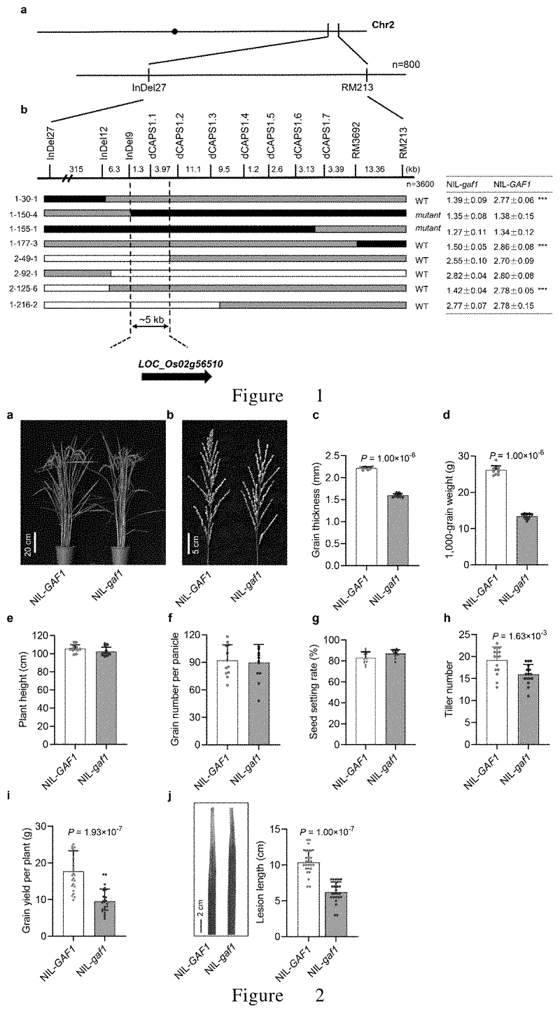

The inventors screened genetic materials with grain filling defects in the field and obtained a mutant with abnormally incomplete filling, which was named gaf1 (grain aberrant and incomplete filling 1). Genetic analysis revealed that this trait is a single trait controlled by a recessive gene. In order to further study the phenotypic traits of gaf1, it was continuously backcrossed with NIP for multiple generations to construct a near-isogenic line (NIL), NIL-GAF1 and NIL-gaf1 (FIG. 1).

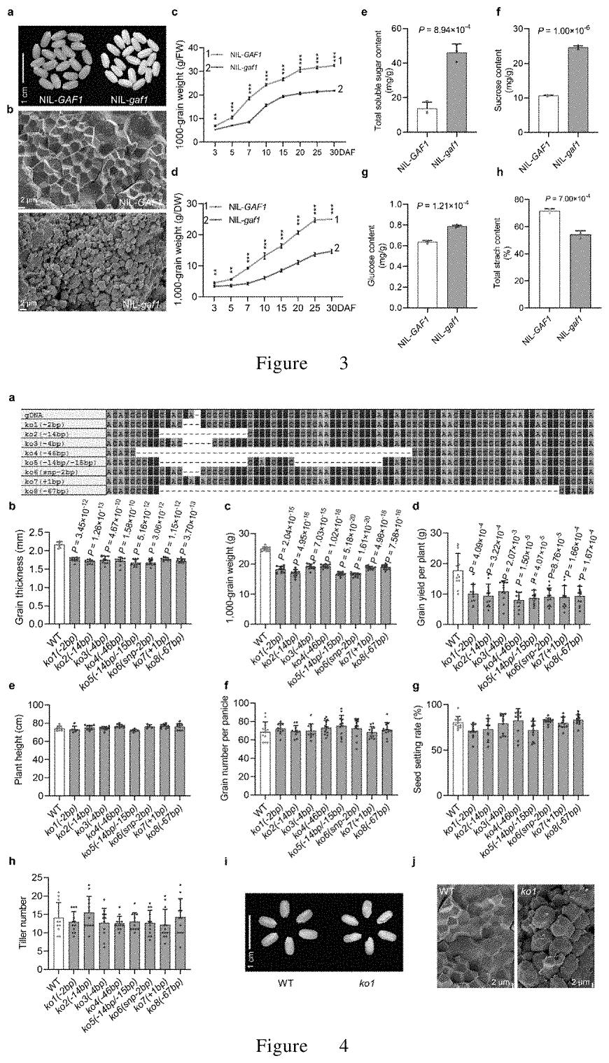

By observing the phenotypes, it was found that NIL-gaf1 exhibited typical grain filling defects (FIG. 2a-b): grain thinning at maturity (FIG. 2c), diminished transparency, significantly decreased 1000-grain weight (FIG. 2d), and severely reduced plant yield (FIG. 2i). However, there were no differences in other agronomic traits such as plant height (FIG. 2e), grain number per panicle (FIG. 2f), seed-setting rate (FIG. 2g), and the tiller number (FIG. 2h), indicating that gaf1 is a key factor that only affects grain filling but not other agronomic traits. By further observation of starch morphology, the inventors found that compared with wild-type NIL-GAF1, the starch granules of NIL-gaf1 were abnormally loose accumulated and irregular in shape, and the total starch was also significantly reduced (FIG. 2, FIG. 3a-b). During the process (0 DAF-30 DAF), both grain weight and grain filling rate of NIL-gaf1 were significantly decreased (FIG. 3c-d). Additionally, in NIL-gaf1, soluble sugar accumulated (FIG. 3e-h) and the resistance to Xanthomonas oryzae pv. oryzae increased, showing resistance to bacterial diseases (FIG. 2j).

In order to further study the regulatory genes of gaf1, the inventors constructed a fine-mapping population by crossing NIL-GAF1 and NIL-gaf1, and finally mapped it in the location of about 5 kb between the markers InDel9 and DCAPS1.2 through 8 key exchange individuals. A detailed sequencing analysis was carried out on this positioning interval, and it was found that there are many nucleotide mutation sites in this interval, including SNP, deletion and so on. Concerning the gene structure, only the front part of the coding region of the gene LOC_Os02 g56510 and the promoter region of the gene are in the 5 kb interval; In terms of sequence differences, the main mutation sites in this region are as follows: Exon1 (TG), Exon3 (GC), Exon7 (1 bp deletion), promoter region (29 bp deletion). Since neither of the two SNPs changed the amino acid sequence (nonsense mutation), the deletion of 1 bp is the cause of the phenotype of the gene mutation.

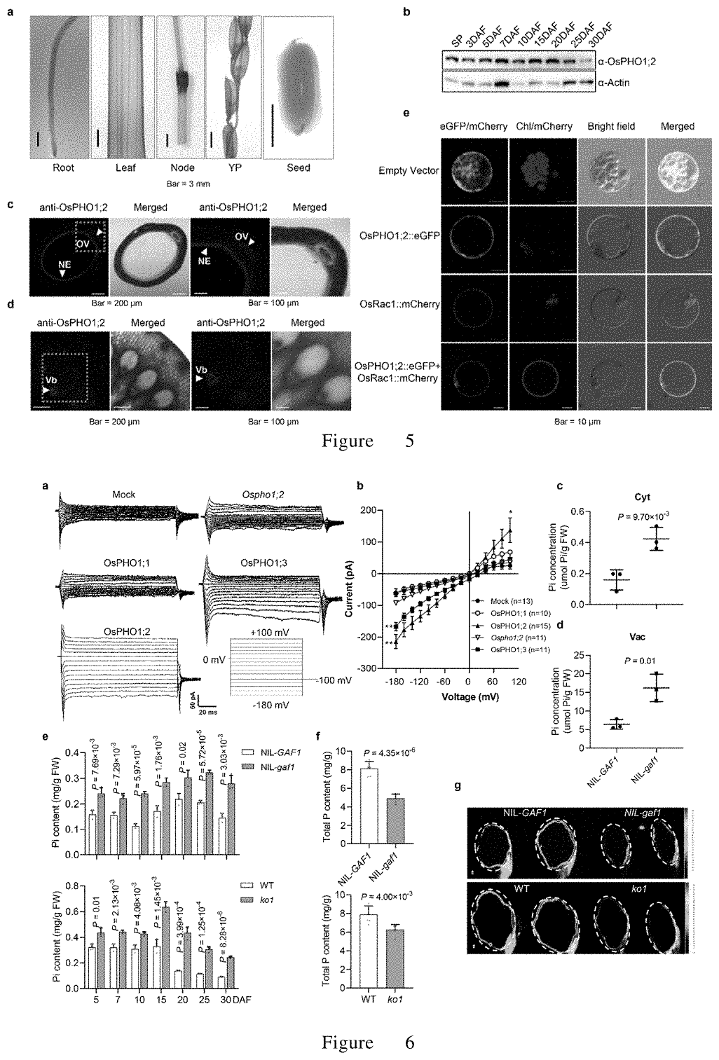

In order to further investigate the pathogenic mutation of gaf1, the inventors used the CRISPR/Cas9 gene editing system to knock out the candidate gene OsPHO1;2, with 8 mutant alleles ko1-ko8 with different mutation types isolated. The difference between the sequences of the corresponding knockout regions are as shown in the figure (FIG. 4a). Agronomic traits of all these mutant alleles were then followed up and showed that, as gaf1, 8 different mutant alleles had severely reduced grain weight (FIG. 4c) and significantly thinner grain thickness (FIG. 4b), resulting in a significant decrease in 1000-grain weight and yield (FIG. 4c-d), while did not affect other agronomic traits such as plant height (FIG. 4e), grain number per panicle (FIG. 4f), the tiller number (FIG. 4h), grain length and grain width, and seed-setting rate (FIG. 4g) and so on (FIG. 4). The inventors randomly selected one of the mutant alleles, ko1, as a follow-up study. The phenotype at maturity was further observed, and there was no significant difference in plant height and panicle shape, while the grain filling saturation was significantly reduced, and the light transmittance was extremely poor (FIG. 4i). Scanning electron microscopy results also showed that in the mutants, the accumulation of starch granules was loose and the starch morphology was severely irregular (FIG. 4j).

Therefore, OsPHO1;2 is the GAF1 functional gene that regulates grain filling in rice.

### Example 2. OsPHO1;2 is a Tissue-Specific Membrane Transporter

The specific function of a gene is closely related to its expression and localization. Therefore, the inventors studied and analyzed the expression pattern and subcellular localization of OsPHO1;2. First, the expression pattern of OsPHO1;2 was analyzed at the transcriptomic level. It was found that OsPHO1;2 was highly expressed mainly in roots, nodes and developing seeds, and this specific expression pattern corresponds to the generation of the grain filling phenotype of gaf1 (FIG. 5a). Importantly, OsPHO1;2 was highly expressed in dehulled seeds during the grain filling process (from the spikelet stage to 30 days after pollination), and gradually decreased by the seed maturation period (30DAF) (FIG. 5b). Immediately, the inventors then performed immunofluorescence detection on the early grain-filling node (node I) and hulled seeds with OsPHO1;2-specific antibody by immunofluorescence technology, so as to observe the localization pattern more accurately. The results showed that in the first section (node I), the fluorescence signal of OsPHO1;2 was detected, and a strong signal was detected in vascular bundle (Vb), indicating that OsPHO1;2 was specifically expressed in the vascular bundle; In addition, more interestingly, OsPHO1;2 was detected in the dehulled seeds with very strong fluorescence signals in nucellar epidermis (NE) of the maternal tissue and the ovary vascular (OV) of the seeds (FIG. 5c-d), with the same results shown in multiple replicates to be tested, and similar results were obtained in the pOsPHO1;2::GUS transgenic line. Reports showed that nucleolar epidermis (NE) and ovary vascular (OV) tissues to be the key “gates” in seeds that mediate nutrients from maternal tissue (pericarp) into daughter tissue (endosperm) (Krishnan and Dayanandan, 2003). Therefore, the inventors speculate that OsPHO1;2 may be involved in mediating the transport of Pi from the pericarp to the endosperm.

Subsequently, the inventors studied the subcellular localization pattern of OsPHO1;2. First, by transient transfection assay of leaf sheath protoplasts in rice, fused construct of OsPHO1;2 and YFP was transiently transformed into protoplasts to observe the fluorescence signal. The results revealed that compared with the empty vector, OsPHO1;2 showed obvious localization signal on the cell membrane. Besides, after co-transfection with the membrane-localized Marker protein, OsPHO1;2 could completely merge with OsRac1 (FIG. 5e). Therefore, OsPHO1;2 is a membrane-localized protein. In addition, the results of OsPHO1;2 cell membrane localization are also confirmed in onion.

Thus, OsPHO1;2 is a membrane-localized phosphorus transporter specifically expressed in the nucleolar epidermis (NE) and vascular bundle (Vb).

### Example 3. OsPHO1;2 is a Two-Way Phosphorus Transporter which Mainly Conveys Phosphorus to the Extracellular

It has been proposed that PHO1;2 is an inorganic phosphorus transporter that mediates Pi transport in root-stem, but its specific transporting properties have not been reported in either arabidopsis or rice. Notably, gaf1/ospho1;2 mutants showed dwarfing and weak growth at the seedling stage, but after about 5 weeks of planting in the field, its plant type quickly returned to normal, and the plant height increased at the mature stage, with no difference compared to wild type (FIG. 2). It indicated that the phosphorus transport in root-stem is not the main function of OsPHO1;2, and the regulation of grain filling during seed development is its main function.

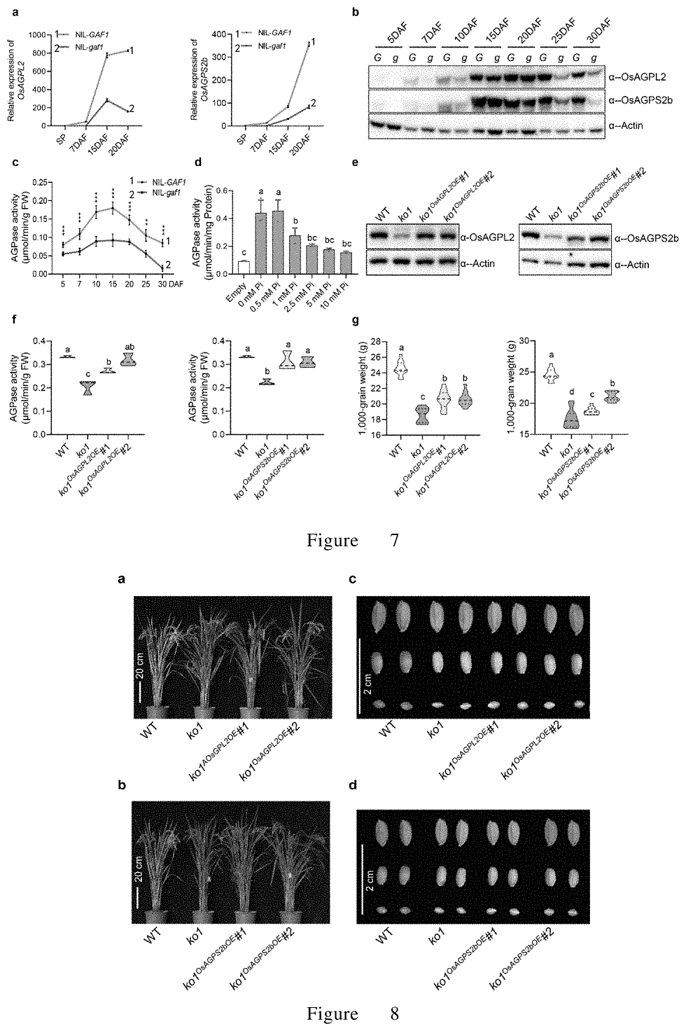

The inventors explored the phosphorus transport functions of OsPHO1;2 in different systems. First, in yeast, the full-length CDS of OsPHO1;2 can successfully complement the yeast phosphorus transport deletion of mutant EY917 (pho84Δ, pho87Δ, pho89Δ, pho90Δ, pho91Δ). Therefore, it is proved that OsPHO1;2 is indeed an inorganic phosphorus transporter. Subsequently, the inventors detected the transport activity of OsPHO1;2 in mammalian cells (HEK293T) using patch-clamp technique. OsPHO1;1, OsPHO1;2, Ospho1;2, OsPHO1;3 were expressed separately in a mammalian cell line (HEK293T), and current-voltage changes were recorded (FIG. 6a). The results revealed that OsPHO1;2 showed strong Pi-in and Pi-out activities, mainly Pi-out activities, while the mutant Ospho1;2 of OsPHO1;2 lost all transport activities. The transport activities of OsPHO1;1 and OsPHO1;3 was also not detected except partial Pi-out activity remains in OsPHO1;3 (FIG. 6a-b). Therefore, OsPHO1;2 is the first two-way phosphorus transporter identified in plants and mainly conveys phosphorus to the extracellular.

In order to further explore the mechanisms of Pi redistribution and Pi balance regulated by OsPHO1;2, it was firstly found at the seedling stage that in the gaf1 mutant, the Pi in the roots accumulated, while the Pi in the stem decreased, regardless of whether it was Pi-deficient or Pi-sufficient. Thus, OsPHO1;2 mutation inhibits Pi transport from root to shoot at the seedling stage. In addition, ³¹P NMR results at the seedling stage showed that in young roots, both the Pi in cytoplasm (Cyt) and the Pi in the vacuole (Vac) were significantly accumulated in mutants (FIG. 6c-d), which also excluded that OsPHO1;2 is involved in the flow and distribution of Pi between vacuole and cytoplasm. At the same time, it was also proved that OsPHO1;2 has exudation-activity, and its mutation leads to the loss of exudation-activity, resulting in Pi accumulation. Subsequently, the inventors detected the Pi levels of all tissues above the ground, and found that the Pi in node I, glumes and dehulled seeds increased, while the Pi in flag leaves and other leaves decreased (FIG. 6g), suggesting that OsPHO1;2 is involved in the redistribution of Pi from seeds to leaves. To further confirm this idea, the inventors tracked the entire filling process, and the results showed that from 5DAF to 30DAF, the Pi in the mutants accumulated significantly (FIG. 6e). Therefore, the mutation of OsPHO1;2 resulted in the inability of Pi exudation to the nutritive organ (leaf) and failed to finish Pi redistribution. Also, the total phosphorus was significantly reduced in mutants (FIG. 6f), possibly because the high Pi in seeds feedback-inhibited the synthesis of phytic acid (PA) or other forms of organic phosphorus or feedback-inhibited the process of total phosphorus metabolism.

In conclusion, OsPHO1;2 is a two-way exudation-dominated phosphorus transporter and its mutation leads to accumulation of Pi in seeds.

### Example 4. Accumulation of Pi Inhibits the Activity of Amylosynthetase

In order to further explore the relationship between Pi content and grain filling, the inventors analyzed the relevant characteristics of kernel starch synthesis. First, samples at the grain filling stage (spikelet, 7DAF, 15DAF, 20DAF) were collected to detect the transcriptional expression levels of starch synthesis-related genes. The analysis of ADP pyrophosphorylase (AGPase), starch synthase (SS), granule-bound starch synthase (GBSS), branching enzyme (BE) and debranching enzyme (DBE) and other enzymes found that many key genes related to starch synthesis showed a down-regulation trend in mutants (FIG. 7a), especially OsAGPL2 and OsAGPS2b, with their protein expressions also significantly down-regulated (FIG. 7b). In addition, the enzymatic activities and gene expressions of other starch-related enzymes were significantly down-regulated. In particular, AGPase is an important rate-limiting enzyme in the process of starch synthesis, which catalyzes G-1-P and ATP to generate ADP-Glc and PPi, and this reaction is a reversible reaction.

Combining with the fact that in gaf1 mutants, the content of Pi significantly accumulated, AGPase activity decreased during the whole filling process, and high Pi could inhibit AGPase activity, the inventors believe that AGPase may be an important effector in the regulation of grain filling mediated by OsPHO1;2. To verify this conjecture, the inventors detected the enzymatic activity of AGPase during the whole grain filling process, and found that the enzymatic activity of AGPase in mutants decreased significantly from 3DAF to 30DAF, corresponding to the accumulation of Pi during the grain filling process (FIG. 7c). Subsequently, by prokaryotically expressing AGPase in E. coli, it was found that high Pi levels could significantly inhibit the enzymatic activity of AGPase (FIG. 7d). In addition, NIL-GAF1 and NIL-gaf1 suspension cell lines were used to further verify the inhibitory effect of Pi on AGPase, and the results showed that excessive Pi in the medium significantly inhibited the expression levels of OsAGPL2 and OsAGPS2b. Taken together, excess Pi negatively affects both AGPase activity and expression, which may be responsible for the reduced starch synthesis and grain filling defects in OsPHO1;2 mutants.

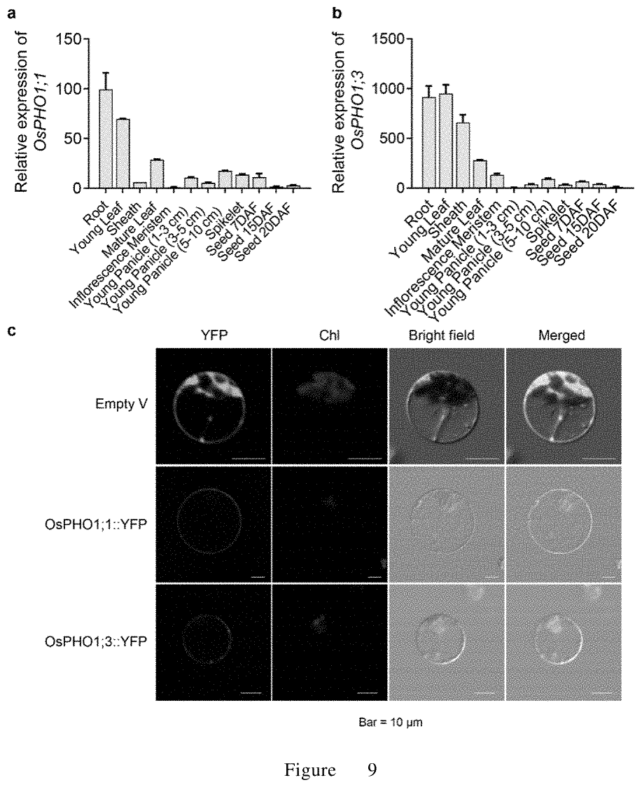

The inventors speculate that OsPHO1;2 affects the enzymatic activity of AGPase by regulating the inorganic phosphorus content in the seed endosperm, thereby promoting or inhibiting the downstream starch synthesis process. After the deletion of this gene, due to the loss of the redistribution and transport of inorganic phosphorus (mainly conveys phosphorus to the extracellular), the accumulation of inorganic phosphorus in seeds cannot be used effectively, which inhibits the enzymatic activity of AGPase, and finally inhibits the process of starch synthesis, resulting in the phenotype of grain filling defect. In order to further verify the speculation, the inventors overexpressed AGPase in mutants by genetic methods, artificially increased its enzyme activity, and then observed its phenotype to see if it could restore or partially restore the filling phenotype of gaf1 to explain the functional mechanisms of OsPHO1;2. OsAGPL2 and OsAGPS2b were overexpressed in the ko1 mutant, respectively, to screen the positive homozygous line AGPase-OE/ko1. Phenotypes of the two mutants were observed and analyzed at the grain filling and the maturity, respectively. The results showed that, compared with ko1, expression of complementary lines ko1^{OsAGPL2}^{_{OE}} and ko1^{OsAGPS2b}^{_{OE}} firstly recovered to the same level as the wild-type WT, and the AGPase activity also recovered to a certain level (FIG. 7e-f). At maturity, by observing phenotypes, it was found that the complementary lines ko1^{OsAGPL2}^{_{OE}} and ko1^{OsAGPS2b}^{_{OE}} were in an intermediate state, significantly different from the mutant ko1 but slightly worse than the wild-type WT, with main performance of significantly earlier heading stage and maturation stage than that of ko1 (FIG. 8a-b). Besides, after fully matured, it was observed that the complementary lines ko1^{OsAGPL2}^{_{OE}} and ko1^{OsAGPS2b}^{_{OE}} were significantly better than ko1 in grain shape and grain filling (FIG. 8c-d). The inventors statistically analyzed the agronomic traits and found that, compared with ko1, the grain weight of the complementary line ko1^{OsAGPL2}^{_{OE}} was restored by about 15%, while the grain weight of the complementary line ko1^{OsAGPS2b}^{_{OE}} was restored by about 10%-20% (FIG. 7g). Therefore, overexpression of AGPase gene can partially restore the grain filling defect of ko1, which also confirms that OsPHO1;2 regulates rice grain filling with appropriate AGPase enzyme activity. This also implies that the increase in yield can be achieved by enhancing AGPase activity to promote grain filling in the production process.

### Example 5. OsPHO1;2 in Rice PHO1 Family Specifically Regulates Grain Filling

The rice PHO1 family has three members: OsPHO1;1, OsPHO1;2 and OsPHO1;3. Studies found that ospho1;2 can respond to phosphorus deficiency, with reduced transport of Pi from roots to shoots, Pi accumulation in roots and reduced Pi content in stems after mutation, while ospho1;1 and ospho1;3 do not respond to Pi (Secco et al., 2010). Therefore, among the PHO1 family, OsPHO1;2 plays a major role in the transport of inorganic phosphorus. The inventors also studied other two genes, OsPHO1;1 and OsPHO1;3. First, the expression patterns of OsPHO1;1 and OsPHO1;3 were explored. The results showed that OsPHO1;1 was mainly highly-expressed in roots and leaves. Similarly, OsPHO1;3 was also highly expressed in roots, stems and leaves. Interestingly, both OsPHO1;1 and OsPHO1;3 showed very low or almost no expression in reproductive organs such as panicles and seeds (FIG. 9a-b). This expression pattern was significantly different and differentiated from OsPHO1;2. However, same as OsPHO1;2, both OsPHO1;1 and OsPHO1;3 are also membrane-localized proteins (FIG. 9c). The results of transport activities also showed that, besides OsPHO1;3 with weaker exudation-activity, the transport activities of OsPHO1;1 and OsPHO1;3 were weaker than those of OsPHO1;2. Also, evolutionary analysis showed that OsPHO1;1 and OsPHO1;3 have a close relationship with AtPHO1;2 in Arabidopsis thaliana, but obviously differentiated from OsPHO1;2, which also determines the specific function of OsPHO1;2 in the PHO1 family.

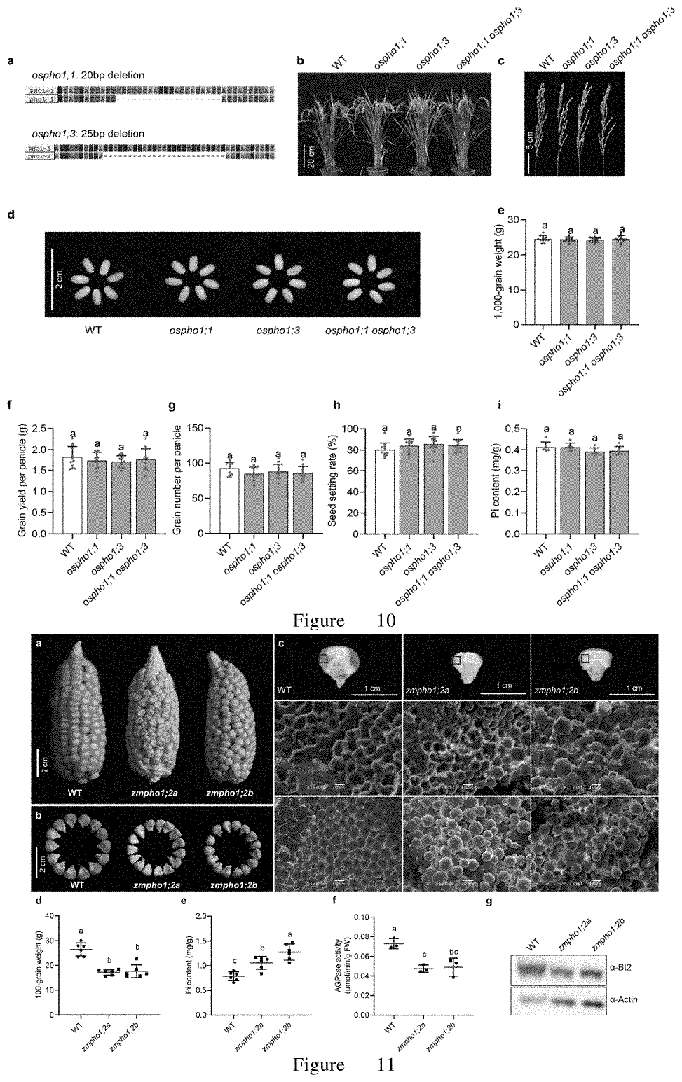

In order to further study the functions of OsPHO1;1 and OsPHO1;3 in rice, the inventors constructed knockout mutants of OsPHO1;1 and OsPHO1;3 by CRISPR/Cas9 gene editing system (FIG. 10a), including a single mutation ospho1;1, a single mutation ospho1;3 and a double mutation ospho1;1 ospho1;3. At maturity, by observing the phenotypes, it was found that, whether it was a single mutation or double mutations, ospho1;1, ospho1;3 and ospho1;1 ospho1;3, compared with the wild type, there were no significant differences in morphology of plants (FIG. 10b), panicle types (FIG. 10c) and grain shapes (FIG. 10d). Further statistical analysis found that the 1000-grain weight (FIG. 10e), plant weight (FIG. 10f), number of grains per ear (FIG. 10g), grain length and grain width, seed setting rate (FIG. 10h) and other agronomic traits were not significantly different from the wild type (FIG. 10), that is, ospho1;1, ospho1;3 and ospho1;1 ospho1;3 had no phenotype changes. In addition, the measurement of inorganic phosphorus in seeds also exhibited no change in phosphorus of ospho1;1, ospho1;3 and ospho1;1os pho1;3 (FIG. 10i). Therefore, combined with the previous transport activity results, transport activities could not be detected neither in OsPHO1;1 nor in OsPHO1;3 (FIG. 6). That is, OsPHO1;1 and OsPHO1;3 were not involved in the long-distance transport of inorganic phosphorus, the redistribution of phosphorus and the regulation of grain filling. Here, in the rice PHO1 family, OsPHO1;2 with exudation activity specifically regulates grain filling and phosphorus redistribution in rice.

### Example 6. ZmPHO1;2 in Maize Regulates Filling of Crop Kernels and Pi Redistribution

Grain filling is an important physiological process and agronomic trait. The inventors speculate that OsPHO1;2, a very important grain filling regulatory gene identified by the present disclosure, may also be a very conserved gene. The inventors compared the PHO1;2 genes of important crops in production, such as rice (Rice), maize (Maize), wheat (Triticum aestivum), sorghum (Sorghum bicolor), millet (Setaria italica), and so on, and found that there are two homologous genes ZmPHO1;2a and ZmPHO1;2b in maize, one homologous gene in both sorghum and millet, while there are 9 homologous genes with close similarities in wheat, possibly due to the huge genome of wheat. By protein sequence aligning of these homologous genes and constructing a phylogenetic tree, the inventors found that OsPHO1;1 and OsPHO1;3 are far from OsPHO1;2 and its homologous genes, which may also be one of the reasons that the OsPHO1;2 exerts functions peculiarly and differentiates functionally. Secondly, homologous genes of OsPHO1;2 in other crops are highly similar to the sequences of OsPHO1;2 in rice, especially in the important crops such as wheat and maize. It implies that PHO1;2 is of great significance in agricultural production and natural evolution.

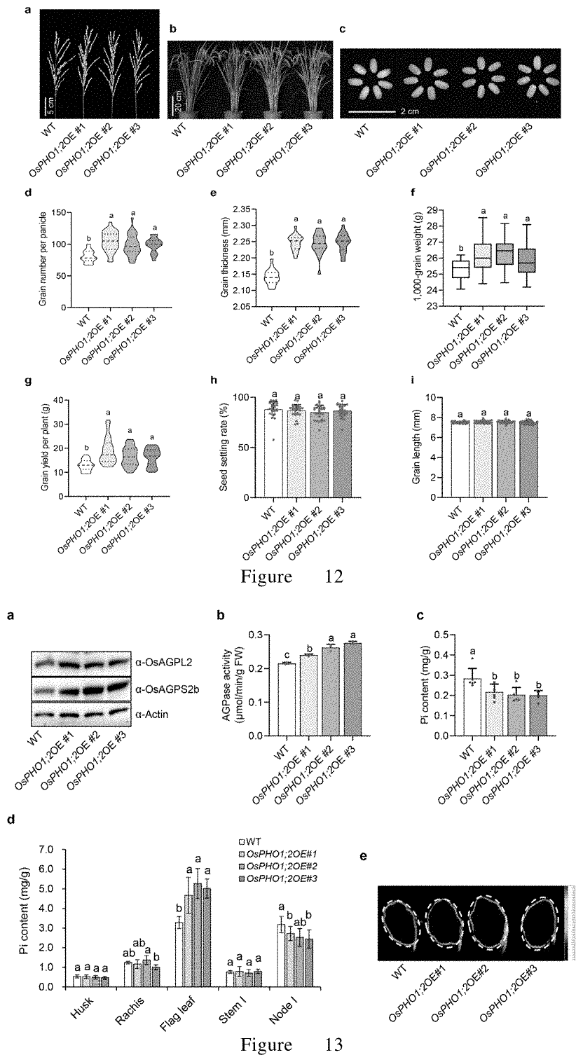

In order to further verify the conservation of PHO1;2 in crops, the inventors selected maize as the object of study. After the two homologous genes in maize, ZmPHO1;2a and ZmPHO1;2b knockouted by CRISPR/Cas9, the wild-type maize inbred line C01 was transformed by the knockouted construct to screen homozygous mutant generations. The homozygous mutant alleles were screened among the mutation types, and one mutant allele was randomly selected for research. After the mutant was self-bred for 2-3 generations of homozygosity, phenotype of the mutants were observed. At maturity, the inventors observed and analyzed phenotypes of kernels and female ears in maize. The results showed that, compared with the wild type, there were no significant differences in shape, size and compact arrangement of kernels between the female ears of zmpho1;2a and zmpho1;2b, but there were extremely significant differences in the kernels of zmpho1;2a and zmpho1;2b with narrowed and shortened kernels, irregular shrinkage, extremely poor transmittance, reduced and shriveled fullness, showing a typical filling defect phenotype (FIG. 11a). Further observation of cross-sections of kernels in the wild-type, zmpho1;2a and zmpho1;2b showed that the starches also changed significantly. In the mutants, with abnormally reduced transparency, almost all starches were opaque starch granules (FIG. 11b). Simultaneously, scanning electron microscopy results showed that in the wild-type WT, starch granules were packed in a regular shape and compact in transparent region of the edge, and packed compact in a spherical shape in opaque region of the central. However, in the zmpho1;2a and zmpho1;b mutants, Both in edge transparent area and in opaque region of the central, regular-shaped and compact starch granules could not be observed, especially the starch granules in the marginal area seemed to be consistent with the central area, with different sizes and loose packing (FIG. 11c), suggesting that in zmpho1;2a and zmpho1;2b mutants of the maize, starch synthesis was also abnormal. The result leads to a significant drop in kernel weight of about 35%. In order to study how ZmPHO1;2 in maize regulates grain filling, similarly, by analogy with the rice model to verify whether the two crops have the same regulation pattern, the inventors analyzed the expression and enzyme activity of AGPase in maize. The results showed that in zmpho1;2a and zmpho1;2b mutants of the maize, the expression of AGPase (Bt2 in maize) was down-regulated (FIG. 11g), and the AGPase activity during the grain filling was also significantly decreased by about 45% (FIG. 11f). By combining the severely accumulated inorganic phosphorus in endosperm (FIG. 11e), the inventors believe that ZmPHO1;2 in maize also regulates grain filling in maize through a mechanism similar to that in rice.

### Example 7. Overexpression of OsPHO1;2 can Significantly Promote Grain Filling and Improve Rice Yield and Pi Utilization

As mentioned above, OsPHO1;2 is a gene that positively regulates grain filling in rice. In order to further explore its potential application, the inventors constructed a 35S promoter-driven OsPHO1;2 overexpressed plant to study its phenotype. Three homozygous overexpressed lines were randomly selected, and agronomic traits, for example plant type, were analyzed at maturity. The results showed that the overexpressed lines were significantly thicker than the wild type in terms of plant type at maturity, with larger panicles and stronger transmittance of kernels, all of which showed improved traits (FIG. 12a-c). Further statistical analysis showed that 1000-grain weight was significantly increased in OsPHO1;2 overexpressed lines (FIG. 12f), with significantly increased yield per plant (FIG. 12g). Interestingly, grain thickness (FIG. 12e) of the mutant was also significantly different from that of wild type, indicating that OsPHO1;2 overexpression makes grain filling more substantial. In addition, the tiller number and grain number per panicle also increased (FIG. 12d), but the grain length, grain width and seed-setting rate showed no effect (FIG. 12h, i). Therefore, overexpression of OsPHO1;2 can significantly increase plant yield.

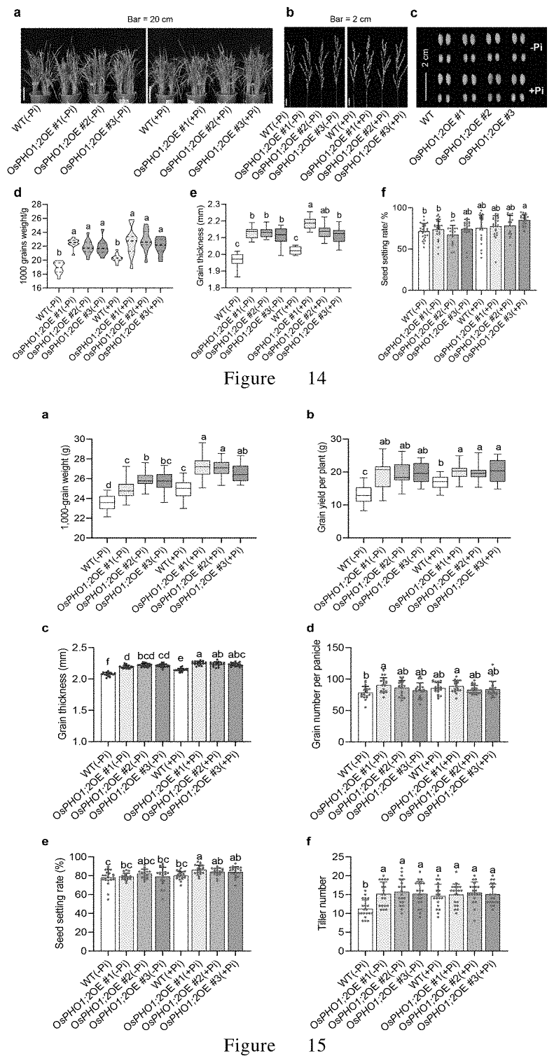

Subsequently, the inventors analyzed the AGPase activity and the distribution of inorganic phosphorus in the OsPHO1;2 overexpressed line. First, the AGPase activities of OsPHO1;2 overexpressed lines were measured at grain filling, and it was found that the enzyme activities in the overexpressed lines also increased (FIG. 13b), along with the increase of OsAGPL2 and OsAGPS2b protein expression (FIG. 13a), indicating that overexpression of OsPHO1;2 increased plant yield by increasing AGPase activity to promote grain filling. Secondly, same tissues, such as brown rice, husk, rachis, node I, stem I, flag leaf, and other tissues, were sampled to measure the content of inorganic phosphorus. The results showed that, unlike the mutant gaf1/ko1, the inorganic phosphorus of the overexpressed lines were significantly decreased in matured kernels and the Pi contents in nodes with a role in distribution were also significantly decreased (FIG. 13c), while the inorganic phosphorus in flag leaves were significantly increased, with no significant difference in Pi of other tissues such as internodes of cob, glumes, and so on (FIG. 13d-e). These results indicate that OsPHO1;2 overexpression promotes the redistribution of Pi, that is, excess inorganic phosphorus in seeds can be redistributed to nutritive organs such as flag leaves after exduation, promoting photosynthesis and producing more nutrients into kernels, ultimately increasing plant yield. Therefore, overexpression of OsPHO1;2 can significantly increase plant yield and promote the redistribution and recycling of phosphorus.

### Example 8. The Application of OsPHO1;2 in Rice

The concentration of inorganic phosphorus that can be directly absorbed by plants is extremely low in soil, about 2-10 μM. In order to ensure normal plant growth and stable high yield of crops, a large amount of phosphorus fertilizer must be applied in the field to ensure sufficient phosphorus concentration for plant absorption and utilization. However, the application of a large amount of chemical fertilizers not only increases economic costs but also causes environmental pollution, which is contrary to the sustainable development of green agriculture. OsPHO1;2 overexpression can significantly increase plant yield and promote the redistribution and recycling of phosphorus, allowing more Pi to return to nutritive tissues for example flag leaves, so as to achieve the goal of high phosphorus utilization. The inventors speculate that OsPHO1;2 can also resist low-phosphorus stress and maintain a good growth state under low-phosphorus conditions. First, the inventors obtained soil with extremely low phosphorus concentration (4.7 ppm Pi) from Nanjing Agricultural University, and used pot-growing method in greenhouse to verify the speculation of inventors. The experiment was divided into two groups, one with very low phosphorus fertilizer (+Pi) and another with no phosphorus fertilizer (−Pi). Except for the variables of phosphorus fertilizer, other conditions were kept the same, such as nitrogen fertilizer, phosphorus fertilizer, temperature, light, and other conditions. Plants were transplanted into pots after about one month of field growth, with 6 treatment replicates and 3 biological replicates per line per treatment. During the grain filling, the inventors observed that due to phosphorus deficiency in soil, in the treatment of no Pi, the wild-type WT exhibited phosphorus-deficient traits, such as: reduced tillers, late heading, withered and yellow leaves, and straight leaves. However, when the overexpression line was treated with no phosphorus in low phosphorus soil, its tolerance to phosphorus deficiency was obviously better than that of the wild type, with significantly increased tillers, less yellow leaves, and earlier heading than that of the wild type (FIG. 14a). This indicates that overexpression of OsPHO1;2 can enhance the tolerance to phosphorus deficiency and efficiently utilize phosphorus in soil to maintain normal growth of plants. At the same time, under the treatment of normal phosphorus in low phosphorus soil, the wild type was recovered. However, the growth of the overexpression line was still better than that of the wild type. At maturity, the inventors performed statistical analysis on the phenotypic traits of each treated line. The results showed that under the no Pi condition, the seeds were wrinkled, small, with decreased seed setting rate and undernourished, and the application of P fertilizer was significantly better than the no Pi condition, with the growth of seeds relieved. However, as for the OsPHO1 overexpressed line, especially under no Pi condition, it still showed excellent grain-filling properties with obvious resistance to the defect of extremely low phosphorus and efficient utilization of the existing tiny amount of phosphorus to maintain growth and seed development, although it is slightly weaker than the P-containing treatment group (FIG. 14b-c). Further statistical analysis of agronomic traits showed that in terms of grain weight, the overexpressed lines showed an ideal grain weight phenotype regardless of whether phosphorus fertilizer was applied, while in the wild type, grain weight decreased significantly and grain filling was severely inhibited, compared with that under the treatment of adding phosphorus. The grain weight in the wild type also decreased significantly, but changed little in the overexpressed lines (FIG. 14d). Subsequently, the results of measuring grain thickness also showed that the grain thickness of the overexpressed lines was significantly higher than that of the wild-type WT (FIG. 14e). Among the overexpressed lines, the grain thickness in no Pi group was slightly smaller than that in the Pi-containing group. The grain thickness in the wild-type Pi-containing group was slightly higher than that in the no-phosphorus group, but the difference was statistically significant (FIG. 14). Other traits such as grain length and grain width were not significantly different (FIG. 14f). These results showed that overexpression of OsPHO1;2 can significantly improve the tolerance to low phosphorus and efficiently utilize phosphorus in soil to maintain good grain-filling characteristics and high yield of plants, suggesting that OsPHO1;2 can improve rice yield and reducing the use of phosphorus fertilizer at the same time.

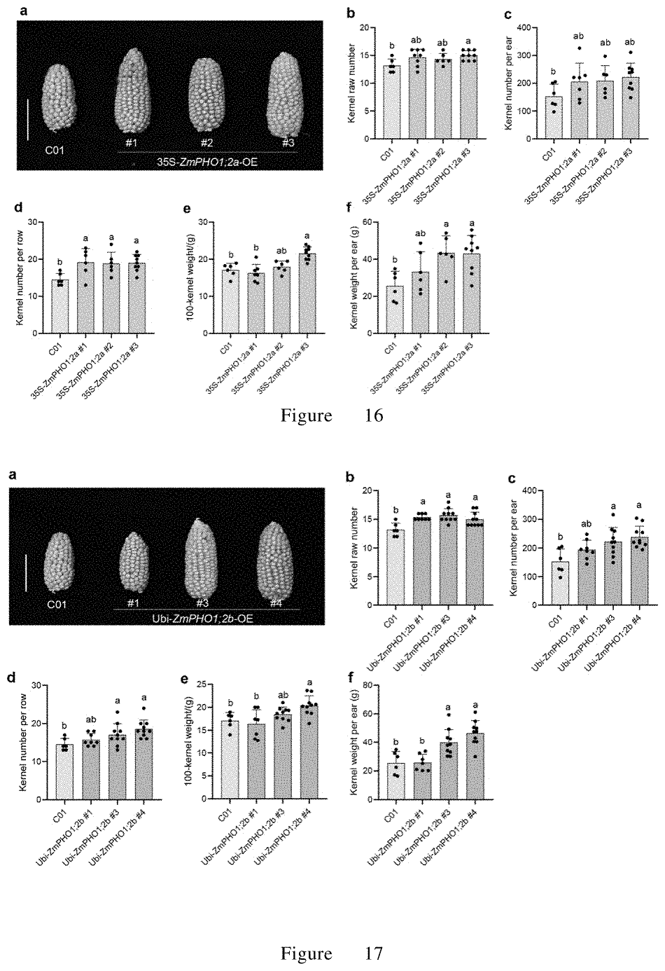

In addition, the inventors also carried out phosphate fertilizer treatment experiments in the field under normal conditions to further explore the value of OsPHO1;2 application. In the field under normal conditions (Shanghai Songjiang Base), the same experiments were designed, that is, normal soil with phosphate fertilizer (+Pi) and no phosphate fertilizer (−Pi), with nitrogen and potassium fertilizers and other conditions kept the same. At maturity, statistical analyses were performed for phenotypic as well as agronomic traits. First, the inventors observed that the grain weight and yield per plant of wild-type WT in two treatments of −Pi and +Pi were extremely significantly reduced under no phosphorus conditions (FIG. 15a-b). This is because phosphorus deficiency leads to undernutrition of plants. On the contrary, the grain weight and yield per plant of the OsPHO1;2 overexpressed lines were significantly higher than those of the wild type, especially the yield of the overexpressed lines increased by 49% under the no phosphorus condition. Besides, compared with the control group with the application of phosphorus fertilizer, yield of the overexpressed lines was not significantly different (FIG. 15a-b). Since OsPHO1;2 is a grain filling regulatory gene, the inventors further analyzed the grain filling of all lines and treatments. First, in terms of grain filling degree (grain thickness), the grain filling was inhibited when phosphorus was deficient in WT, with severely decreased grain thickness, which are significantly different from the +Pi group, and the grain thickness of the overexpressed lines was almost equivalent between the two treatments (FIG. 15c); with grain length and grain width unchanged (FIG. 15). These results indicate that overexpression of OsPHO1;2 can also efficiently utilize soil phosphorus to maintain plant growth and development in normal soil, and maintain high-yield traits at maturity. In addition, the tiller number and grain number per panicle of the wild type in −Pi treatment decreased because of phosphorus deficiency (FIG. 15d, f), but the seed setting rate did not change (FIG. 15e). However, the overexpressed lines still remained strong advantages of similar characteristics to the +Pi group. Therefore, overexpression of OsPHO1;2 significantly improved phosphorus use efficiency (PUE), increased rice yield under low phosphorus conditions, and decreased the input of phosphorus fertilizer, providing a new target option for crop yield increase and green sustainable development.

In the present disclosure, ZmPHO1;2 in maize also regulates grain filling in maize and Pi redistribution and utilization by a conservative mechanism similar to OsPHO1;2 in rice, while overexpression of OsPHO1;2 in rice significantly improved phosphorus use efficiency (PUE) and increased rice yield. It can be expected that overexpression of ZmPHO1;2 in maize can also significantly increase the yield of maize, which will be an important discovery for increasing the yield of crops. The study of PHO1;2 gene provides a good guidance and a target option for reducing the use of phosphorus fertilizer, protecting the environment and increasing yield in agricultural production.

### Example 9. PHO1;2 Gene has a Regulating Function on Kernels of Maize

To further verify the effect of PHO1;2 gene in maize, the inventors constructed over-expressed vectors driven by 35S promoter and Ubi promoter according to two homologous genes ZmPHO1;2a and ZmPHO1;2b in maize, respectively. The vectors were transformed into the C01 inbred lines and the related phenotypes were observed in T2 generation after stable inheritance.

The results showed that compared with the wild type C01, three overexpressed lines all showed larger panicles (FIG. 16a). After harvest, agronomic traits were further statistically analyzed. The kernel row number, kernel number per ear, kernel number per row, 100-kernel weight, and kernel weight per ear of maize were significantly increased, especially in 35S-ZmPHO1:2a#3, wherein the increase is extremely significant (FIG. 16b-f).

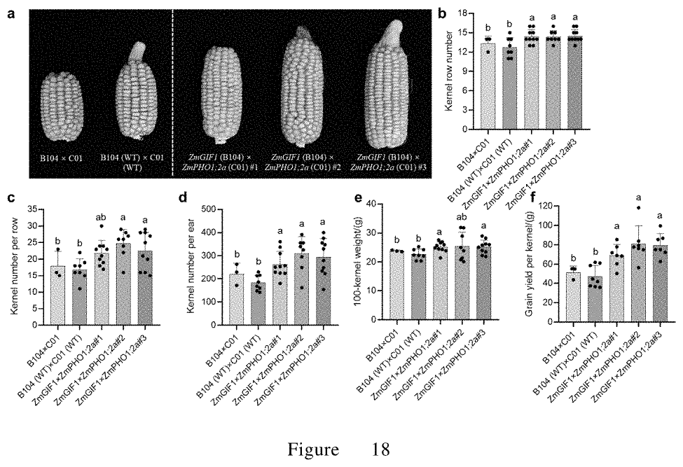

On the other hand, in ZmPHO1;2b overexpressed lines driven by Ubi promoter, similar trends were also observed, that is, overexpression of ZmPHO1;2b leads to bigger ears, and simultaneously increases final grain weight and yield (FIG. 17a-f).

These results showed that, consistent with the results in rice, overexpressing PHO1;2 gene in maize can also promote grain filling and yield improvement, further explaining that PHO1;2 is widely applicable in gramineous crops to regulate grain filling and contribute to yield.

### Example 10. PHO1;2 Gene has a Regulating Function on Kernels of Maize

As described in Example 6, the effect of yield increase in maize is due to PHO1;2 optimization of Pi balance in kernels, with reduced accumulation of Pi, thus maintaining a high filling rate. GIF1 gene is also a key regulator of grain filling, encoding a cytoderm sucrose invertase, which converts sucrose to glucose and fructose. The absent of GIF1 causes grain filling defects and overexpression of this gene in rice and maize can promote yield improvement (Wang, E. et al. (2008), Nat Genet 40, 1370-1374; Li, B. et al. (2013), Plant Biotechnol J 11, 1080-1091; 200610117721.6). However, the improvement of yield is not a single goal of crop improvement. Reducing the phosphorus content of kernels, especially the content of phytic acid, has always been an important goal for improvement. That is because phytic acid is an important anti-nutrient molecule, which will reduce the absorption and utilization of other nutrients and simultaneously, because of the lack of phytase, the animal phytic acid is hard to digest after eating. The release of animal phytic acid into the environment results in environmental pollution and water eutrophication. The inventors found that simultaneous overexpression of two filling genes PHO1;2 and ZmGIF1 caused a significant increase in yield of maize and a decrease in phosphorus content.

The inventors constructed the ZmGIF1 overexpressed lines of maize B104 background, and hybridized with ZmPHO1;2a and ZmPHO1;2b overexpressed lines with C01 background after stable inheritance, using wild-type B104 and C01 as control, with two transgenic negative plants of the two genes also as control to observe the phenotype in F1 generation.

The results showed that compared with F1 in the control, the F1 line with two overexpressed genes had significantly better growth, and larger ears. After the statistical analysis of agronomic traits at maturity, simultaneous overexpression of ZmGIF1 and ZmPHO1 can significantly increase the yield of maize and various agronomic indicators, including kernel number per ear, kernel raw number, 100-kernel weight, and other indicators, with an increase of more than 90% (FIG. 17a-f). The combination of PHO1;2 and ZmGIF1 has the advantage of not only the improvement of single traits, but also an important value for application by greatly optimizing and improving the yield of phosphorus content.

### Example 11. Screening Methods

Cells: In a mammalian cell line (HEK293T), OsPHO1;2 was overexpressed.

Testing group: In cultured cells overexpressing OsPHO1;2, the candidate substance was administered;

Control group: In cultured cells overexpressing OsPHO1;2, the candidate substance was not administered;

The expressions or activities of OsPHO1;2 in the testing group and the control group were detected and compared. If the expression or activity of OsPHO1;2 in the testing group is statistically higher (eg. increase by 30% or higher) than that in the control group, the candidate substance can be used as the potential substance for improving plant filling traits.

Each reference provided herein is incorporated by reference to the same extent as if each reference was individually incorporated by reference. In addition, it should be understood that based on the above teaching content of the disclosure, those skilled in the art can practice various changes or modifications to the disclosure, and these equivalent forms also fall within the scope of the appended claims.

1. A method for improving crop traits or preparing crops with improved traits, comprising: up-regulating the expression or activity of PHO1;2 in crops; the PHO1;2 comprises homologues thereof;
wherein, the improved crop traits are selected from the group comprising: (i) promoting filling of crop kernels; (ii) increasing crop yield or biomass, (iii) promoting two-way phosphorus transport effect which mainly conveys phosphorus to the extracellular, regulating intracellular phosphorus accumulation; (iv) enhancing ADP pyrophosphorylase activity; (v) increasing the utilization rate of phosphorus in crops; (vi) improving the tolerance of crops to a low-phosphorus environment.

2. The method according to claim 1, wherein the up-regulation of the expression or activity of PHO1;2 comprises: overexpressing exogenous PHO1;2 in crops; comprising:
introducing a PHO1;2 gene or an expression construct or vector comprising the gene into the crops;
using an enhancer or a tissue-specific promoter to improve the expression of PHO1;2 gene in crops;
increasing PHO1;2 gene expression in crops with enhancers; or
decreasing histone-methylation level of the PHO1;2 gene and increasing its expression level.

3. The method according to claim 1, wherein, it also comprises up-regulating the expression or activity of GIF1 in crops, comprising:
introducing a GIF1 gene or an expression construct or vector comprising the gene into the crops;
using an enhancer or a tissue-specific promoter to improve the expression of GIF1 gene in crops; or
increasing GIF1 gene expression in crops with enhancers.

4. The method according to claim 1, wherein the increase of crop yield or biomass comprises: increasing grain weight, tiller number, grain number and grain thickness, and/or promoting thickening of the crops.

5. The method according to claim 1, wherein the two-way phosphorus transport effect which mainly conveys phosphorus to the extracellular comprises extracellular phosphorus transport and intracellular phosphorus transport; or
the two-way phosphorus transport effect which mainly conveys phosphorus to the extracellular comprises: promoting the redistribution and recycling of phosphorus; it further comprises transferring the extra intracellular phosphorus of the crop kernels out of the cells and redistributing it to the nutritive organs.

6. The method according to claim 1, wherein, the crops are cereal crops or the PHO1;2 or homologues thereof are derived from cereal crops; the cereal crops comprise Gramineous plants.

7. The method according to claim 6, wherein, the gramineous plants comprises: rice (Oryza sativa), maize (Zea mays), millet (Setaria italica), barley (Hordeum vulgare), wheat (Triticum aestivum), millet (Panicum miliaceum), broomcorn (Sorghum bicolor), rye (Secale cereale), oats (Avena sativa L).

8. The method according to claim 1, wherein the amino acid sequence of PHO1;2 polypeptide is selected from the following groups:
(i) a polypeptide having the amino acid sequence shown in any one of SEQ ID NO: 1-3;
(ii) a polypeptide derived from the polypeptide of (i) by substitution, deletion or addition of one or several residues in the amino acid sequence of any one of SEQ ID NO: 1-3 and having the function of regulating said traits;
(iii) a polypeptide having the amino acid sequence with more than 80% identity to the amino acid sequence of any one of SEQ ID NO: 1-3 and having the function of regulating said traits;
(iv) an active fragment of the polypeptide having the amino acid sequence shown in any one of SEQ ID NO: 1-3; or
(v) a polypeptide derived from the amino acid sequence shown in any one of SEQ ID NO: 1-3 with a tag or an enzyme-cleavage sequence added at N-terminus or C-terminus; or a signal polypeptide fused at N-terminus.

9. The method according to claim 3, wherein, the amino acid sequence of GIF1 polypeptide is selected from the following groups:
(i) a polypeptide having the amino acid sequence shown in SEQ ID NO: 4;
(ii) a polypeptide derived from the polypeptide of (i) by substitution, deletion or addition of one or several amino acid residues in the amino acid sequence of SEQ ID NO: 4 and having the function of regulating traits;
(iii) a polypeptide having the amino acid sequence with more than 80% identity to the amino acid sequence of SEQ ID NO: 4 and having the function of regulating traits;
(iv) an active fragment of the polypeptide having the amino acid sequence shown in SEQ ID NO: 4; or
(v) a polypeptide derived from the amino acid sequence shown in SEQ ID NO: 4 with a tag or an enzyme-cleavage sequence added at N-terminus or C-terminus; or a signal polypeptide fused at N-terminus.

10. A crop or cells thereof, wherein, it expresses an expression cassette of exogenous PHO1;2 or homologues thereof; the expression cassette comprises: a promoter, an encoding gene of PHO1;2 or its homologues, a terminator.

11. The crop or cells thereof according to claim 10, wherein it also expresses an expression cassette of exogenous GIF1 or homologues thereof; the expression cassette comprises: a promoter, an encoding gene of GIF1 or its homologues, a terminator.

12. The crop or cells thereof according to claim 10, wherein, the amino acid sequence of PHO1;2 polypeptide is selected from the following groups:
(i) a polypeptide having the amino acid sequence shown in any one of SEQ ID NO: 1˜3;
(ii) a polypeptide derived from the polypeptide of (i) by substitution, deletion or addition of one or several amino acid residues in the amino acid sequence of any one of SEQ ID NO: 1˜3 and having the function of regulating traits;
(iii) a polypeptide having the amino acid sequence with more than 80% identity to the amino acid sequence of any one of SEQ ID NO: 1˜3 and having the function of regulating traits;
(iv) an active fragment of the polypeptide having the amino acid sequence shown in any one of SEQ ID NO: 1˜3; or
(v) a polypeptide derived from the amino acid sequence shown in any one of SEQ ID NO: 1˜3 with a tag or an enzyme-cleavage sequence added at N-terminus or C-terminus; or a signal polypeptide fused at N-terminus.

13. The crop or cells thereof according to claim 11, wherein, the amino acid sequence of GIF1 polypeptide is selected from the following groups:
(i) a polypeptide having the amino acid sequence shown in SEQ ID NO: 4;
(ii) a polypeptide derived from the polypeptide of (i) by substitution, deletion or addition of one or several amino acid residues in the amino acid sequence of SEQ ID NO: 4 and having the function of regulating traits;
(iii) a polypeptide having the amino acid sequence with more than 80% identity to the amino acid sequence of SEQ ID NO: 4 and having the function of regulating traits;
(iv) an active fragment of the polypeptide having the amino acid sequence shown in SEQ ID NO: 4; or
(v) a polypeptide derived from the amino acid sequence shown in SEQ ID NO: 4 with a tag or an enzyme-cleavage sequence added at N-terminus or C-terminus; or a signal polypeptide fused at N-terminus.

14. A method for directional screening crops with improved traits, the method comprises: analyzing PHO1;2 gene expression or PHO1;2 protein activity in crops; if the PHO1;2 gene expression or PHO1;2 protein activity in crops to be tested is higher than the average value of the crops, it has: (i) high kernels (seeds) filling level; (ii) high yield or biomass, (iii) excellent two-way phosphorus transport effect which mainly conveys phosphorus to the extracellular, regulating intracellular phosphorus accumulation; (iv) enhanced ADP pyrophosphorylase activity; (v) increased utilization rate of phosphorus in crops; (vi) improved tolerance of crops to a low-phosphorus environment; wherein, the PHO1;2 gene comprises homologues thereof.

15. The method according to claim 14, wherein, it further comprises: analyzing GIF gene expression or GIF protein activity in crops; if the GIF gene expression or GIF protein activity in crops to be tested is higher than the average value of the crops, it indicates that the crops have improved traits.

16. A method for screening substances for improving crop traits, wherein the method comprises:
(1) adding candidate substance to the system expressing PHO1;2;
(2) detecting the system to observe the expression or activity of PHO1;2; if the expression or activity is up-regulated, then the candidate substance can be used as the substance to improve traits of crops;
wherein, the improved crop traits are selected from the following group comprising: (i) promoting filling of crop kernels; (ii) increasing crop yield or biomass, (iii) promoting two-way phosphorus transport effect which mainly conveys phosphorus to the extracellular, regulating intracellular phosphorus accumulation; (iv) enhancing ADP pyrophosphorylase activity; (v) increasing the utilization rate of phosphorus in crops; (vi) improving the tolerance of crops to a low-phosphorus environment.

17. The method according to claim 16, wherein the system of (1) also expresses GIF1; and in (2), it further comprises: detecting the system to observe the expression or activity of GIF1; if the expression or activity is also elevated when the expression or activity of PHO1;2 is elevated, then the candidate substance can be used as the substance to improve traits of crops.

18. The method according to claim 14, wherein, the crops are gramineous plants, or the PHO1;2 or homologues thereof are derived from gramineous plants.

19. The method according to claim 18, wherein, the gramineous plants comprises: rice (Oryza sativa), maize (Zea mays), millet (Setaria italica), barley (Hordeum vulgare), wheat (Triticum aestivum), millet (Panicum miliaceum), broomcorn (Sorghum bicolor), rye (Secale cereale), oats (Avena sativa L).


SEQUENCE LISTING (4 sequences)

SEQ ID NO: 1 - protein, 815 aa, Oryza sativa
MVKFSREYEASIIPEWKAAFVDYKRLKKLIKRIKVTRRDDSFAAANAAAAADHLLPPPPAEKEAGGYGFSILDPVRAIAARFSAGQQPSASEDEECPDRGELVRSTDKHEREFMERADEELEKVNAFYTGQEAELLARGDALLEQLRILADVKRILADHAAARRARGLARSRSMPPPPPSSSPPSSVHGSSGRYLLSGLSSPQSMSDGSLELQQAQVSEGAAVADEVMAALERNGVSFVGLAGKKDGKTKDGSGKGRGGGGGGGGGVLQLPATVRIDIPATSPGRAALKVWEELVNVLRKDGADPAAAFVHRKKIQHAEKNIRDAFMALYRGLELLKKFSSLNVKAFTKILKKFVKVSEQQRATDLFSEKVKRSPFSSSDKVLQLADEVECIFMKHFTGNDRKVAMKYLKPQQPRNTHMITFLVGLFTGTFVSLFIIYAILAHVSGIFTSTGNSAYMEIVYHVFSMFALISLHIFLYGCNLFMWKNTRINHNFIFDFSSNTALTHRDAFLMSASIMCTVVAALVINLFLKNAGVAYANALPGALLLLSTGVLFCPFDIFYRSTRYCFMRVMRNIIFSPFYKVLMADFFMADQLTSQIPLLRHMEFTACYFMAGSFRTHPYETCTSGQQYKHLAYVISFLPYFWRALQCLRRYLEEGHDINQLANAGKYVSAMVAAAVRFKYAATPTPFWVWMVIISSSGATIYQLYWDFVKDWGFLNPKSKNRWLRNELILKNKSIYYVSMMLNLALRLAWTESVMKIHIGKVESRLLDFSLASLEIIRRGHWNFYRLENEHLNNVGKFRAVKTVPLPFRELETD

SEQ ID NO: 2 - protein, 553 aa, Zea mays
MAALERNGVSFVGSGLGSKAKKDGGGKQLTGRAAALPATVRIDVPPTSPGRAALKVWEELVNVLRKDGADPAAAFVHRKKVQHAEKSIRDAFLALYRGLDLLNKFSSLNVKAFTKILKKFVKVSEQQRKTDLFSEKVKRSPFSSSDKVLQLADEVECIFSRHFAGNDRKVAMKYLKPQQPRNTHMITFLVGLFTGTFVSLFIIYSVLAHVAGIFSSTGNTAYMEIVYHVFSMFALISLHVFLYGCNLLAWKSSRISHNFIFDFSPSTALTHRDAFLLSASIMCTVVAALVVNLFLSNAGATYANALPGALLLLSAAALFCPFNVFYRSTRYCFMRVMRNIMLSPFYKVLMADFFMADQLTSQIALLRHLEFTGCYFMAGTFTTHAYGSCTSSSQYKNLAYVLSFLPYYWRAMQCLRRYLEEGHDIDQLANAGKYISAMVAAAVRFKYAAAPTPFWMWMVIVSSTGATIYQLYWDFVMDWGFLDLRSKNRWLRDQLILKNKPIYYASMMLNLVLRLAWAESVMKLRLGMVESRLLDFSLASLEIIRRGHWNFYR

SEQ ID NO: 3 - protein, 805 aa, Zea mays
MVKFSREYEASIIPEWKAAFVDYKGLKKLVKRIKIARRDRAARSTSNDHDDATTTTYGFSVLDPVRALASHFNNATPPASPEGGSDDALRSLESDSGELVRATDKHEQEFVERADEELEKVNKFYAAQEADMLARGDALIEQLRILADVKRILADHAAASSRRGRARLARTGGNSSPPSVDGSNSGRHLLSSPFVASSPQSMSDGSVQLQQARVAEGAAVAEEVMAALERNGVSFVGGGLGKAKKDGSGKQLMGRAALLQLPATVRIDIPPTSPGRAALKVWEELVNVLRKDGADPAAAFVHRKKVQHAEKSIRDAFLALYRGLDLLKKFSSLNVKAFTKILKKFVKVSEQHRKGDLFSEKVKRSPFSSSDKVLQLADEVECIFLRHFAGNDRKVAMKYLKPQQPRNTHMVTFLVGLFTGTFVSLFIIYSVLAHVAGIFSSTGNTAYMEIVYHVLSMFALISLHVFLYGCNLSMWKGTRINHNFIFDFSSTALTHRDAFLMSASIMCTVVAALVVNLFLRNAGATYANALPGALLLLSAGVLFCPFNIFYRSTRFCFMRVMRNIVLSPFYKVLMADFFMADQLTSQIPLLRHLEFTGCYFMAETFRTHAYGSCTSSSQYKNLAYVLSFLPYYWRAMQCLRRYLEEGHDMNQLANAGKYVSAMVAAAVRFKYAATPTPFWMWMVIASSTGATIYQLYWDFVMDWGFLNPKSKNFWLRDQLILKNKSIYYASMMLNLVLRLAWAESVMKLRLGMVESRLLDFSLASLEIIRRGHWNFYRLENEHLNNAGKFRAVKTVPLPFRELETD

SEQ ID NO: 4 - protein, 593 aa, Zea mays
MRALVVVSFASACLLLLLQLAGASHVVYNYKDLEAEAAAATDQVPPSIVNPLLRTGYHFQPPKNWINDPNAPMYYKGWYHFFYQYNPKGAVWGNIVWAHSVSRDLINWVALEPALRPSIPGDRYGCWSGSATVLPDGGGPVIMYTGVDHPDINYQVQNVAYPKNVSDPLLREWVKPSHNPVIVPEGGINATQFRDPTTAWRGPGPEQWRLLVGSAAGSSPRGVAYVYRSRDFRRWRRVRRPLHSAATGMWECPDFYPVSKGGAPRAGLETSVPPGPRVKHVLKNSLDLRRYDYYTVGTYHPRAERYVPDDPAGDEHRLRYDYGNFYASKTFYDPAKRRRILWGWANESDSAADDVAKGWAGIQAIPRTVWLDPSGKQLLQWPIEEVEALREKSVTLKNRLIKAGHHVEVTGIQTAQADVEVSFEVSPAALAGAETLDPALAYDAEKLCGVKRADVRGGVGPFGLWVLASANRKERTAVFFRVFKPAAGSDKPVVLMCTDPTKSSLNPNLYRPTFAGFVDTDISNGKISLRSLIDRSVVESFGAGGKTCILSRVYPSLAIGKDARLYVFNNGRAHVKVSRLTAWEMKKPVMNGA